(19) United States
(12) Patent Application Publication
(10) Pub. No.: US 2025/0340577 A1
(43) Pub. Date: Nov. 6, 2025

(54) BETA-LACTOMASE INHIBITORS AND USES THEREOF

(71) Applicants: Case Western Reserve University, Cleveland, OH (US); Universita' degil Syudi di Modena e Reggio Emilia, Modena (MO) (IT)

(72) Inventors: Robert BONOMO, Cleveland, OH (US); Fabio Prati, Modena (MO) (IT); Emilia Caselli, Modena (MO) (IT)

(21) Appl. No.: 18/548,766

(22) PCT Filed: Mar. 2, 2022
(86) PCT No.: PCT/US2022/018522

§ 371 Date: Sep. 1, 2023

Related U.S. Application Data
(60) Provisional application No. 63/155,578, filed on Mar. 2, 2021.
(60) Provisional application No. 63/212,336, filed on Jun. 18, 2021.

(51) Int. Cl. C07F 5/02; A61K 31/397; A61K 31/407; A61K 31/43; A61K 31/545; A61K 31/69

(52) U.S. Cl. CPC C07F 5/025; A61K 31/397; A61K 31/407; A61K 31/43; A61K 31/545; A61K 31/69

(57) ABSTRACT

A pharmaceutical composition for use in treating a bacterial infection in a subject in need thereof includes a β-lactam antibiotic and boronic acid compound as described herein.

### RELATED APPLICATION

This application claims priority from U.S. Provisional Application Nos. 63/155,578, filed Mar. 2, 2021, and 63/212,336, filed Jun. 18, 2021, the subject matter of which are incorporated herein by reference in their entirety.

### GOVERNMENT FUNDING

This invention was made with government support under AI072219 awarded by the National Institutes of Health. The government has certain rights in the invention.

### FIELD OF THE INVENTION

This application relates to compounds for use as β-lactamase inhibitors and particularly to compounds, compositions, and methods for treating microbial or bacterial infections.

### BACKGROUND

Bacterial production of β-lactamases represents the most clinically concerning mechanism of resistance to β-lactam antibiotics in Gram negative bacteria. The emergence of many recent β-lactamase variants (>1600 new enzymes) jeopardizes the efficacy of both the latest developed antibiotics and the combination of β-lactam/β-lactam inhibitor (BLI) (e.g., the formulation ampicillin and clavulanic acid are ineffective against bacteria expressing inhibitor resistant TEM and SHV β-lactamases). At the moment, boronic acids transition state inhibitors (BATSIs) represent a class of BLIs in development, as shown by the advancement of a combination of a boronic acid inhibitor (RPX7009 currently developed by the Medicines Company) with meropenem (a carbapenem), in clinical trials (Clinical Trials Phase 1 registration number NCT01897779, RPX7009 is joined with RPX2014). Carbavance (meropenem/RPX7009) is particularly targeted against KPC (Klebsiella pneumoniae carbapenemase)-producing carbapenem-resistant Enterobacteriaceae (CRE).

α-Amidomethaneboronic acid is a recurring core-structure in biologically active and important boron-containing compounds. α-Amidomethaneboronate unit is the basic structure of peptidoboronic acids, a class of peptidomimetics largely explored to target different clinically relevant proteases. For example, the anticancer Velcade is a dipeptidyl boronic acid (Phe-boroLeu) acting as proteasome inhibitor, while derivatives of the type Val-boroPro or Pro-boroAla have been investigated as dipeptidyl peptidase-4 inhibitors for the treatment of diabetes. The same skeleton is part also of simpler acylamidomethaneboronic acids, reported as subtilisin and α-chymotrypsin inhibitors and used as fluorescent carbohydrate sensors.

### SUMMARY

Embodiments described herein relate to boronic acid compounds or boronic acid transition state inhibitors (BATSIs) and to their use in inhibiting β-lactamase and treating a bacterial infection in a subject in need thereof.

In some embodiments, the boronic acid compound can have the structure of formula (I):

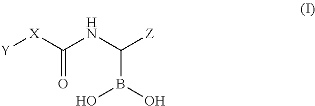

or a pharmaceutically acceptable salt, tautomer, or solvate thereof, wherein:
   X is alkylene or CNOR¹;
   Y is —W—R² or R²;
   Z is H, -alkylene-aryl, -alkylene-heterocyclyl, or -alkylene-heteroaryl, each of which is optionally substituted with one or more R³;
   W is an S, N, O, S(O)ₘN, or alkylene optionally substituted with one or more halogen, alkyl, —COOH, —NH₂, or —OH;
   R¹ is alkyl, -alkylene-OH optionally substituted with —OH, -alkylene-COOH optionally substituted with —OH, -alkylene-O-alkylene-OH, —C(O)-alkyl, —C(O)O-alkyl, or —C(O)O-alkylene-O—C(O)-alkyl;
   R² is heterocyclyl or heteroaryl, each of which is optionally substituted with one or more R⁴;
   each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), or —S(O₂)O⁻;
   each R⁴ is independently halogen, alkyl, —COOH, —NH₂, or —OH;
   each R⁵ is independently H or alkyl;
   m is 0, 1, or 2; and
   if X is CH₂, Y is —W—R².

In some embodiments, R¹ is C₁-C₆ alkyl, —(C₁-C₆ alkylene)-OH optionally substituted with —OH, —(C₁-C₆ alkylene)-COOH optionally substituted with —OH, —(C₁-C₆ alkylene)-O—(C₁-C₆ alkylene)-OH, —C(O)—(C₁-C₆ alkyl), —C(O)O—(C₁-C₆ alkyl), or —C(O)O—(C₁-C₆ alkylene)-O—C(O)—(C₁-C₆ alkyl).

In other embodiments, R¹ is C₁-C₆ alkyl or —(C₁-C₆ alkylene)-COOH optionally substituted with —OH.

In some embodiments, W is S, N, O, or S(O)ₘN; and m is 0, 1, or 2.

In some embodiments, R² is

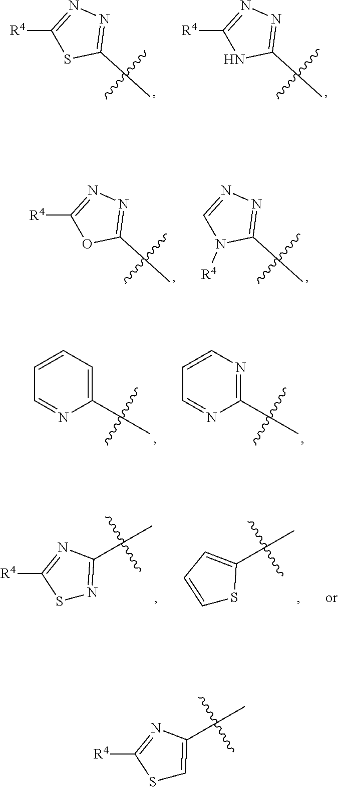

and R⁴ is halogen, alkyl, —COOH, —NH₂, —OH, or absent.

In some embodiments, Z is H, —CH₂-5- to 6-membered aryl, —CH₂-5- to 6-membered heterocyclyl, —CH₂-5- to 6-membered heteroaryl, each of which is optionally substituted with one or more R³.

In some embodiment, each R³ is independently —COOH,

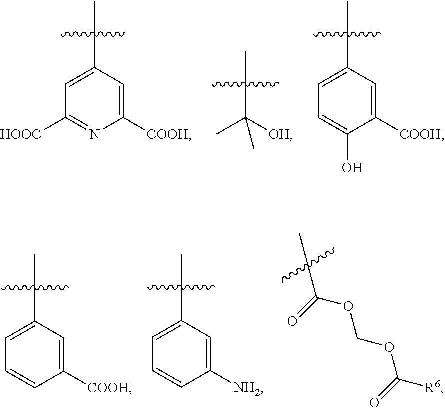

wherein R⁶ is alkyl, such as a C₁-C₆ alkyl, optionally substituted with one or more halogen, —COOH, —NH₂, or —OH.

In other embodiments, Z is H,

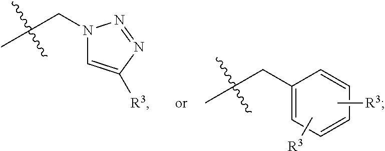

and each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), —S(O₂)O⁻ or absent; and each R⁵ is independently H or alkyl.

In some embodiments, the boronic acid compound can have the structure of formula (II):

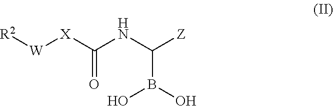

or a pharmaceutically acceptable salt, tautomer, or solvate thereof, wherein:
X is alkylene or CNOR¹;
Z is H, -alkylene-aryl, -alkylene-heterocyclyl, or -alkylene-heteroaryl, each of which is optionally substituted with one or more R³;
W is S, N, O, S(O)ₘN, or alkylene optionally substituted with one or more halogen, alkyl, —COOH, —NH₂, or —OH;
R¹ is alkyl, -alkylene-OH optionally substituted with —OH, -alkylene-COOH optionally substituted with —OH, -alkylene-O-alkylene-OH, —C(O)-alkyl, —C(O)O-alkyl, or —C(O)O-alkylene-O—C(O)-alkyl;
R² is heterocyclyl or heteroaryl, each of which is optionally substituted with one or more R⁴;
each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), or —S(O₂)O⁻;
each R⁴ is independently halogen, alkyl, —COOH, —NH₂, or —OH;
each R⁵ is independently H or alkyl; and
m is 0, 1, or 2.

In some embodiments, R¹ is C₁-C₆ alkyl, —(C₁-C₆ alkylene)-OH optionally substituted with —OH, —(C₁-C₆ alkylene)-COOH optionally substituted with —OH, —(C₁-C₆ alkylene)-O—(C₁-C₆ alkylene)-OH, —C(O)—(C₁-C₆ alkyl), —C(O)O—(C₁-C₆ alkyl), or —C(O)O—(C₁-C₆ alkylene)-O—C(O)—(C₁-C₆ alkyl).

In some embodiments, W is S, N, O, or S(O)ₘN; and m is 0, 1, or 2.

In other embodiments, R¹ is C₁-C₆ alkyl or —(C₁-C₆ alkylene)-COOH optionally substituted with —OH.

In some embodiments, R² is

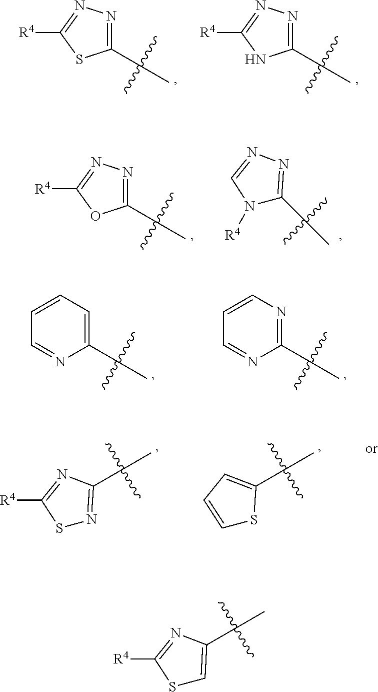

and R⁴ is halogen, alkyl, —COOH, —NH₂, —OH, or absent.

In some embodiments, Z is H, —CH₂-5- to 6-membered aryl, —CH₂-5- to 6-membered heterocyclyl, or —CH₂-5- to 6-membered heteroaryl, each of which is optionally substituted with one or more R³.

In some embodiment, each R³ is independently —COOH,

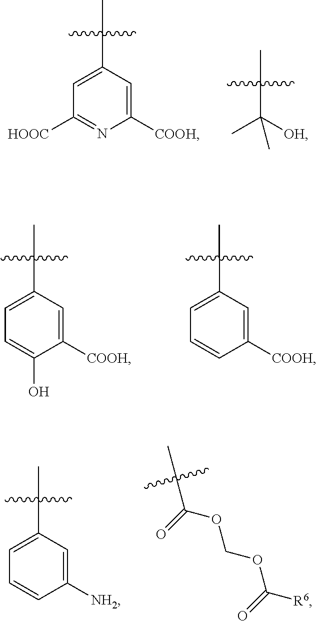

wherein R⁶ is alkyl, such as a C₁-C₆ alkyl, optionally substituted with one or more halogen, —COOH, —NH₂, or —OH.

In some embodiments, Z is H,

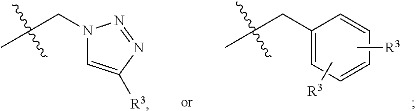

and each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), —S(O₂)O⁻, or absent; and each R⁵ is independently H or alkyl.

In other embodiments, boronic acid compound can have the structure of formula (III):

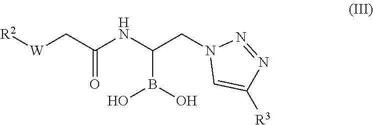

or a pharmaceutically acceptable salt, tautomer, or solvate thereof, wherein:
W is S, N, O, S(O)ₘN, or alkylene optionally substituted with one or more halogen, alkyl, —COOH, —NH₂, or —OH;
R² is heterocyclyl or heteroaryl, each of which is optionally substituted with one or more R⁴;
R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), —S(O₂)O⁻, or absent;
each R⁴ is independently halogen, alkyl, —COOH, —NH₂, or —OH; and
each R⁵ is independently H or alkyl.

In some embodiments, R² is

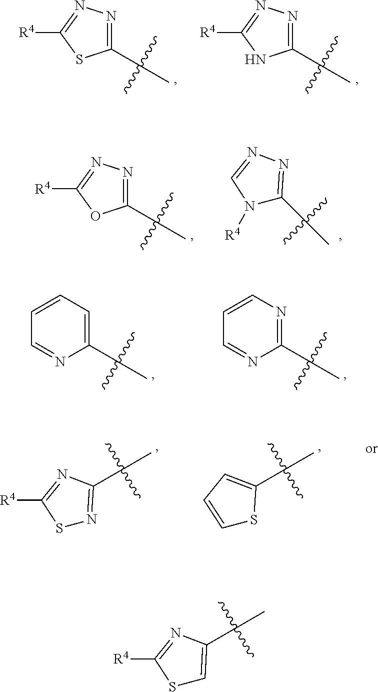

and R⁴ is halogen, alkyl, —COOH, —NH₂, —OH, or absent.

In some embodiments, W is S, N, O, or S(O)ₘN; and m is 0, 1, or 2.

In some embodiment, each R³ is independently —COOH,

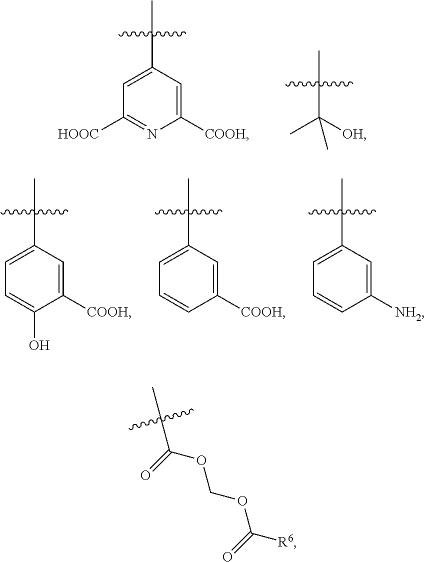

wherein R⁶ is alkyl, such as a C₁-C₆ alkyl, optionally substituted with one or more halogen, —COOH, —NH₂, or —OH.

In other embodiments, the boronic acid compound can have the structure of formula (IV):

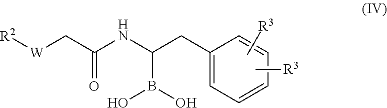

or a pharmaceutically acceptable salt, tautomer, or solvate thereof, wherein:
W is S, N, O, S(O)ₘN, or alkylene optionally substituted with one or more halogen, alkyl, —COOH, —NH₂, or —OH;
R² is heterocyclyl or heteroaryl, each of which is optionally substituted with one or more R⁴;
each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), —S(O₂)O⁻ or absent;
each R⁴ is independently halogen, alkyl, —COOH, —NH₂, or —OH; and
each R⁵ is independently H or alkyl.

In some embodiments, R² is:

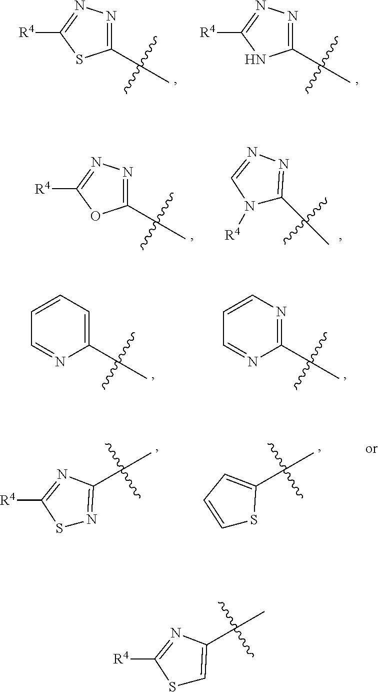

and R⁴ is halogen, alkyl, —COOH, —NH₂, —OH, or absent.

In some embodiments, W is S, N, O, or S(O)ₘN; and m is 0, 1, or 2.

In some embodiments, R³ is —COOH.

In still other embodiment, the boronic acid compound can have the structure of formula (V):

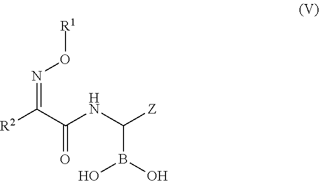

or a pharmaceutically acceptable salt, tautomer, or solvate thereof, wherein:
Z is H, -alkylene-aryl, -alkylene-heterocyclyl, or -alkylene-heteroaryl, each of which is optionally substituted with one or more R³;
R¹ is alkyl, -alkylene-OH optionally substituted with —OH, -alkylene-COOH optionally substituted with —OH, -alkylene-O-alkylene-OH, —C(O)-alkyl, —C(O)O-alkyl, or —C(O)O-alkylene-O—C(O)-alkyl;
R² is heterocyclyl or heteroaryl, each of which is optionally substituted with one or more R⁴;
each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), or —S(O₂)O⁻;
each R⁴ is independently halogen, alkyl, —COOH, —NH₂, or —OH; and
each R⁵ is independently H or alkyl.

In some embodiments, R¹ is C₁-C₆ alkyl, —(C₁-C₆ alkylene)-OH optionally substituted with —OH, —(C₁-C₆ alkylene)-COOH optionally substituted with —OH, —(C₁-C₆ alkylene)-O—(C₁-C₆ alkylene)-OH, —C(O)—(C₁-C₆ alkyl), —C(O)O—(C₁-C₆ alkyl), or —C(O)O—(C₁-C₆ alkylene)-O—C(O)—(C₁-C₆ alkyl).

In other embodiments, R¹ is C₁-C₆ alkyl or —(C₁-C₆ alkylene)-COOH optionally substituted with —OH.

In some embodiments, R² is

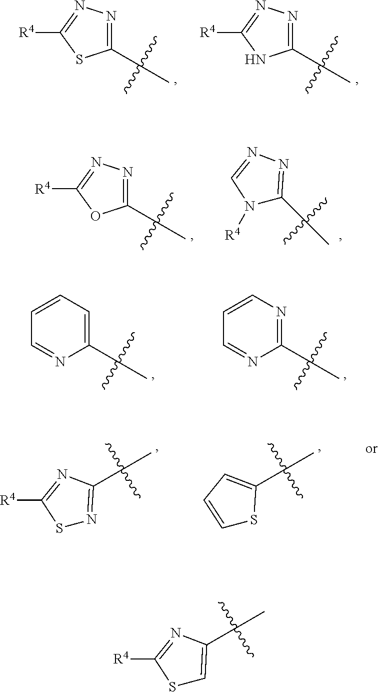

and R⁴ is halogen, alkyl, —COOH, —NH₂, —OH, or absent.

In some embodiments, Z is H, —CH₂-5- to 6-membered aryl, —CH₂-5- to 6-membered heterocyclyl, or —CH₂-5- to 6-membered heteroaryl, each of which is optionally substituted with one or more R³.

In some embodiment, each R³ is independently —COOH,

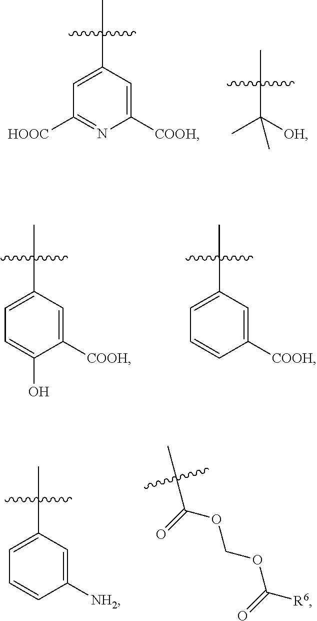

wherein R⁶ is alkyl, such as a C₁-C₆ alkyl, optionally substituted with one or more halogen, —COOH, —NH₂, or —OH.

In some embodiments, Z is H,

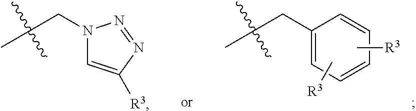

and
each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), or —S(O₂)O⁻ or absent; and each R⁵ is independently H or alkyl.

In some embodiments, the boronic acid compound does not have the structure:

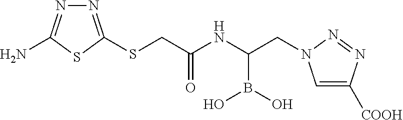 [SMILES: Nc1nnc(SCC(=O)NC(Cn2cc(C(=O)O)nn2)B(O)O)s1]

and a pharmaceutically acceptable salt, tautomer, and solvate thereof.

In some embodiments, the boronic acid compound has a structure of:

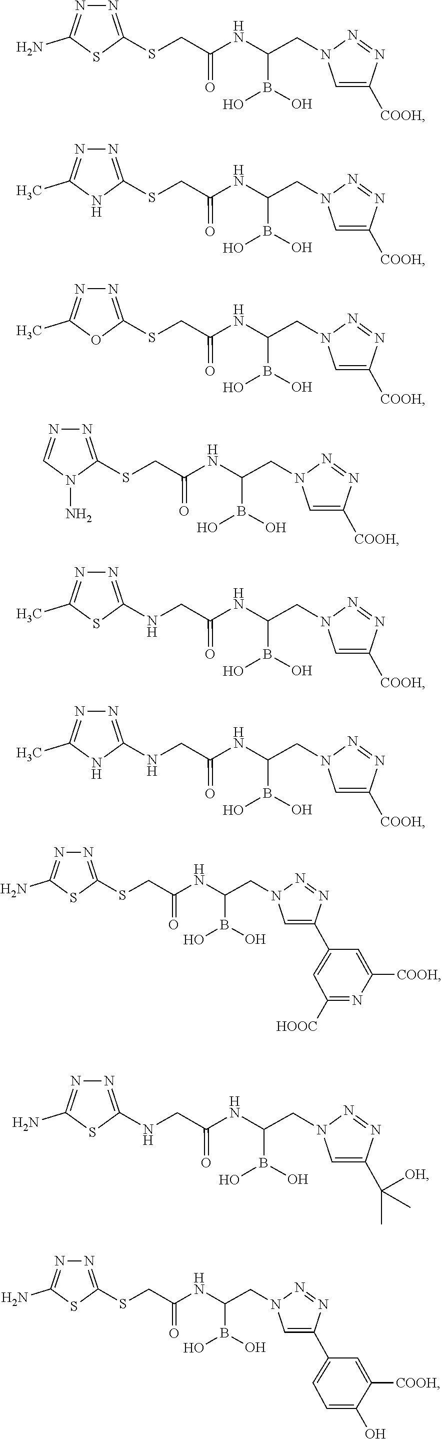

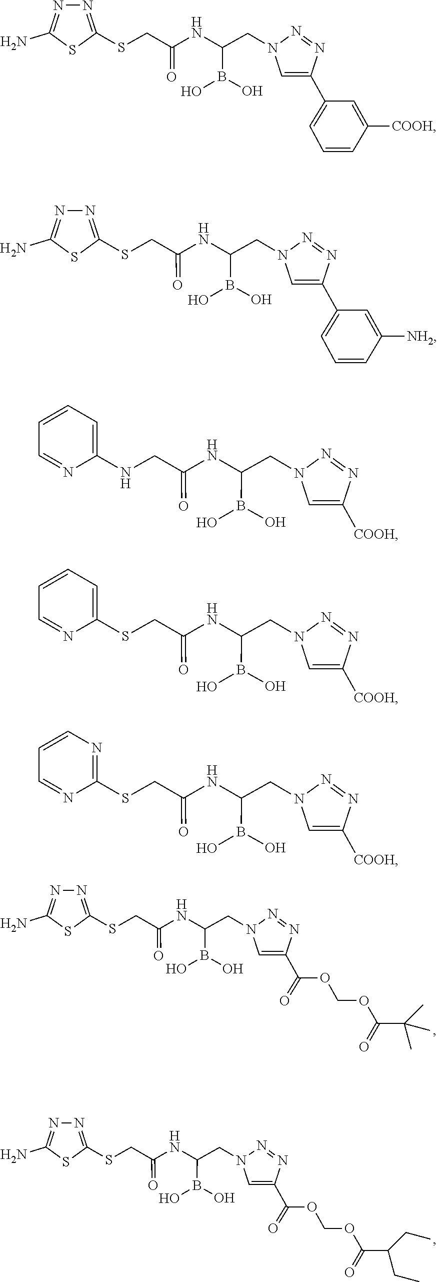

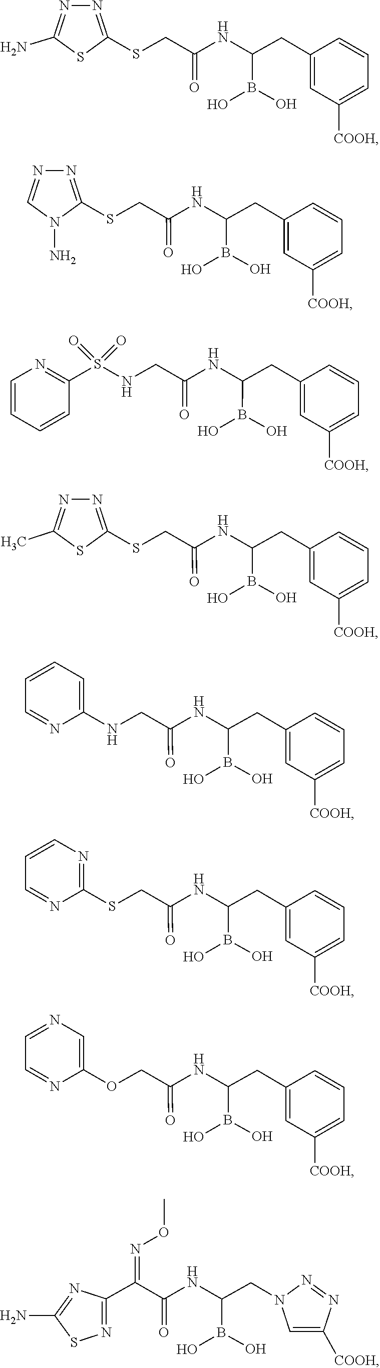

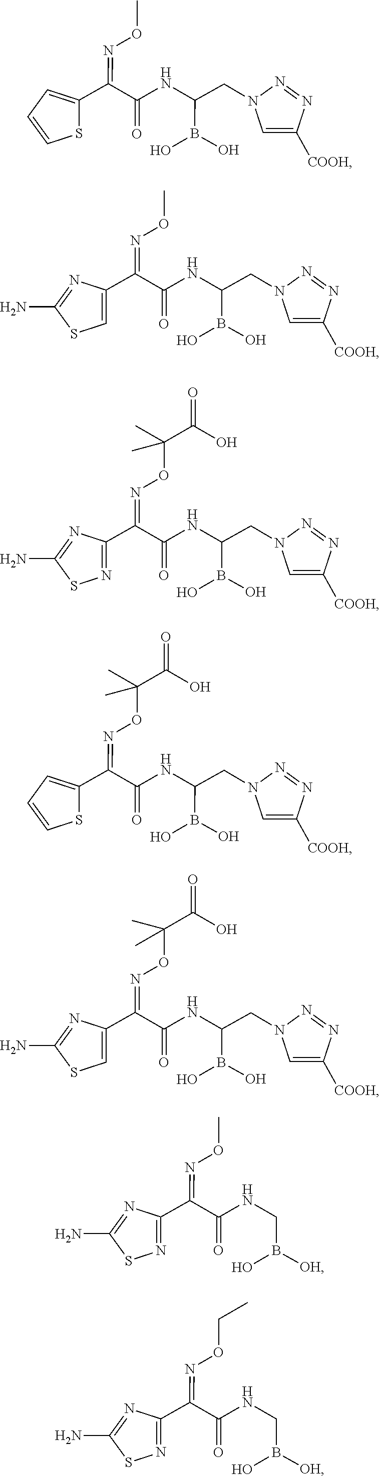

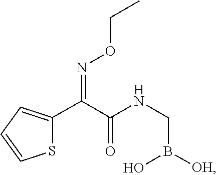 [SMILES: CCO/N=C(\C(=O)NCB(O)O)c1cccs1]

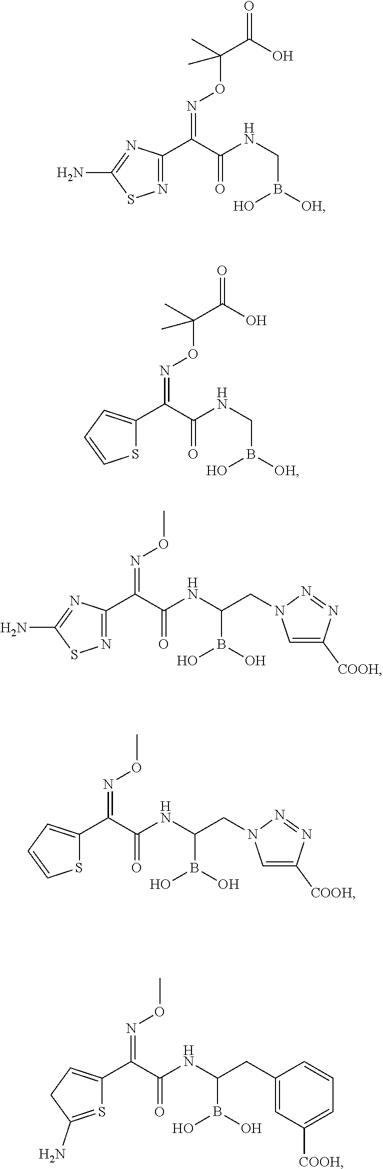

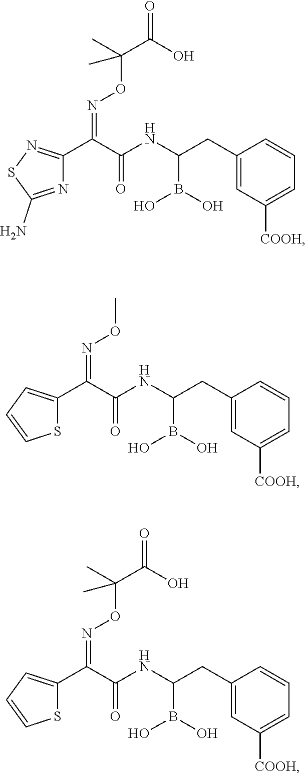 [SMILES: CC(C)(O/N=C(\C(=O)NC(Cc1cccc(C(=O)O)c1)B(O)O)c1nsc(N)n1)C(=O)O ; CO/N=C(\C(=O)NC(Cc1cccc(C(=O)O)c1)B(O)O)c1cccs1 ; CC(C)(O/N=C(\C(=O)NC(Cc1cccc(C(=O)O)c1)B(O)O)c1cccs1)C(=O)O]

or a pharmaceutically acceptable salt, tautomer, or solvate thereof.

Other embodiments described herein relate to a pharmaceutical composition that includes a boronic acid compound or BATSI described herein and a β-lactam antibiotic.

In some embodiments, the β-lactam antibiotic comprises at least one of a penicillin, cephalosporin, penem, carbapenem, or monobactam. For example, the β-lactam antibiotic can include at least one of amoxicillin, ampicillin, azlocillin, mezlocillin, hetacillin, bacampicillin, carbenicillin, sulbenicillin, ticarcillin, piperacillin, mecillinam, methicillin, ciclacillin, oxacillin, cloxacillin, dicloxacillin, nafcillin, cephalothin, cephaloridine, cefaclor, cefadroxil, cefamandole, cefazolin, cephalexin, cephradine, ceftizoxime, cefoxitin, cephacetrile, cefotiam, cefotaxime, cefsulodin, cefoperazone, cefinenoxime, cefmetazole, cephaloglycin, cefonicid, cefodizime, cefpirome, ceftazidime, ceftriaxone, cefpiramide, cefozopran, cefepime, cefuzonam, cefpimizole, cefclidin, cefixime, ceftibuten, cefdinir, cefpodoxime axetil, cefpodoxime proxetil, cefteram pivoxil, cefetamet pivoxil, cefcapene pivoxil, cefditoren pivoxil, cefuroxime, cefuroxime axetil, loracarbacef, latamoxef, CXA-101, imipenem, meropenem, biapenem, panipenem, ertapenem, doripenem, aztreonam, carumonam, and pharmaceutically acceptable salts.

In some embodiments, the β-lactam antibiotic is meropenem and the boronic acid compound or BATSI has the structure

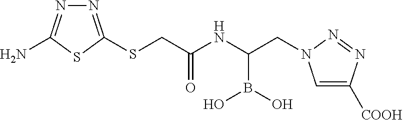 [SMILES: Nc1nnc(SCC(=O)NC(Cn2cc(C(=O)O)nn2)B(O)O)s1]

or a pharmaceutically acceptable salt, tautomer, or solvate thereof.

Still other embodiments described herein relate to a method of inhibiting β-lactamase. The method includes administering to the β-lactamase a boronic acid compound as described herein.

In some embodiments, the β-lactamase is produced by a bacteria and the compound is administered to the bacteria, such as a gram negative bacteria.

Other embodiments described herein relate to a method of treating a bacterial infection in a subject in need thereof. The method includes administering to the subject therapeutically effective amounts of at least one β-lactam antibiotic and a boronic acid compound as described herein. The bacterial infection can a β-lactam antibiotic resistant bacterial infection, for example, a gram negative bacterial infection or β-lactam antibiotic resistant gram negative bacterial infection.

In some embodiments, the β-lactam antibiotic comprises at least one of a penicillin, cephalosporin, penem, carbapenem, or monobactam. For example, the β-lactam antibiotic can include at least one of amoxicillin, ampicillin, azlocillin, mezlocillin, hetacillin, bacampicillin, carbenicillin, sulbenicillin, ticarcillin, piperacillin, mecillinam, methicillin, ciclacillin, oxacillin, cloxacillin, dicloxacillin, nafcillin, cephalothin, cephaloridine, cefaclor, cefadroxil, cefamandole, cefazolin, cephalexin, cephradine, ceftizoxime, cefoxitin, cephacetrile, cefotiam, cefotaxime, cefsulodin, cefoperazone, cefinenoxime, cefmetazole, cephaloglycin, cefonicid, cefodizime, cefpirome, ceftazidime, ceftriaxone, cefpiramide, cefozopran, cefepime, cefuzonam, cefpimizole, cefclidin, cefixime, ceftibuten, cefdinir, cefpodoxime axetil, cefpodoxime proxetil, cefteram pivoxil, cefetamet pivoxil, cefcapene pivoxil, cefditoren pivoxil, cefuroxime, cefuroxime axetil, loracarbacef, latamoxef, CXA-101, imipenem, meropenem, biapenem, panipenem, ertapenem, doripenem, aztreonam, carumonam, and pharmaceutically acceptable salts.

In some embodiments, the β-lactam antibiotic is meropenem and the boronic acid compound or BATSI has the structure

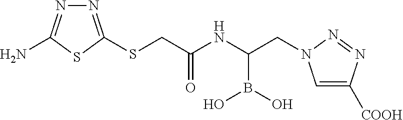 [SMILES: Nc1nnc(SCC(=O)NC(Cn2cc(C(=O)O)nn2)B(O)O)s1]

or a pharmaceutically acceptable salt, tautomer, or solvate thereof.

### BRIEF DESCRIPTION OF THE DRAWINGS

FIG. 1 illustrates the survival curve of mice infected IV with K. pneumoniae Kp1 and treated with cefepime alone or in combination with MB076. Five male C57BL/6 mice per treatment were infected IV via the tail-vein with 2.1×10⁸ CFU/mouse and treated with placebo control (phosphate-buffered saline), cefepime (100 mg/kg/dose), or cefepime and MB076.

FIGS. 2(A-B) illustrate plots showing the mean plasma and tight concentration-time profiles of MB076 after IV single administration to K. pneumoniae AR-BANK #0098 (KPC-2) thigh infected mice.

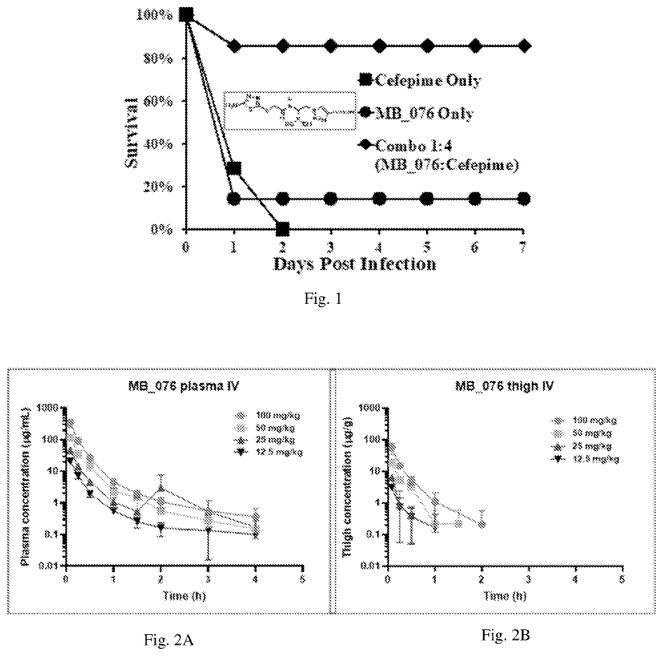

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this application belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.,”, as used herein are non-limiting and are for illustrative purposes only. “Including” and “including but not limited to” are used interchangeably.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “or” as used herein should be understood to mean “and/or”, unless the context clearly indicates otherwise.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

The term “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

Additionally, the salts of the compounds described herein, can exist in either hydrated or unhydrated (the anhydrous) form or as solvates with other solvent molecules. Non-limiting examples of hydrates include monohydrates, dihydrates, etc. Nonlimiting examples of solvates include ethanol solvates, acetone solvates, etc.

The term “solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The compounds and salts described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can be catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

The terms below, as used herein, have the following meanings, unless indicated otherwise:
“Amino” refers to the —NH₂ radical.
“Cyano” refers to the —CN radical.
“Halo” or “halogen” refers to bromo, chloro, fluoro or iodo radical.
“Hydroxy” or “hydroxyl” refers to the —OH radical.
“Imino” refers to the ═NH substituent.
“Nitro” refers to the —NO₂ radical.
“Oxo” refers to the ═O substituent.
“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “C_{w}-C_{z} acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₂-C₁₀ alkenyl, or C₂-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from phenyl (benzene), aceanthrylene, acenaphthylene, acephenanthrylene, anthracene, azulene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}—R_{c} where R_{b} is an alkylene group as defined above and R_{c} is one or more aryl radicals as defined above. Aralkyl radicals include, but are not limited to, benzyl, diphenylmethyl and the like. Unless stated otherwise specifically in the specification, an aralkyl group can be optionally substituted.

“Aralkenyl” or “arylalkenyl” refers to a radical of the formula —R_{b}—R_{c} where R_{b} is an alkenylene group as defined above and R_{c} is one or more aryl radicals as defined above. Unless stated otherwise specifically in the specification, an aralkenyl group can be optionally substituted.

“Aralkynyl” or “arylalkynyl” refers to a radical of the formula —R_{b}—R_{c} where R_{b} is an alkynylene group as defined above and R_{c} is one or more aryl radicals as defined above. Unless stated otherwise specifically in the specification, an aralkynyl group can be optionally substituted.

“Carbocyclyl,” “carbocyclic ring” or “carbocycle” refers to a ring structure, wherein the atoms which form the ring are each carbon. Carbocyclic rings can comprise from 3 to 20 carbon atoms in the ring. Carbocyclic rings include aryls and cycloalkyl. Cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused, bridged, or spiral ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused, bridged, or spiral ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused, bridged, or spiral ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkynyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused, bridged, and spiral ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, aziridinyl, oextanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, pyridine-one, and the like. The point of attachment of the heterocyclyl, heterocyclic ring, or heterocycle to the rest of the molecule by a single bond is through a ring member atom, which can be carbon or nitrogen. Unless stated otherwise specifically in the specification, a heterocyclyl group can be optionally substituted.

“Heterocyclylalkyl” refers to a radical of the formula —R_{b}—Rₑ where R_{b} is an alkylene group as defined above and Rₑ is a heterocyclyl radical as defined above. Unless stated otherwise specifically in the specification, a heterocyclylalkyl group can be optionally substituted.

“Heterocyclylalkenyl” refers to a radical of the formula —R_{b}—Rₑ where R_{b} is an alkenylene group as defined above and Rₑ is a heterocyclyl radical as defined above. Unless stated otherwise specifically in the specification, a heterocyclylalkenyl group can be optionally substituted.

“Heterocyclylalkynyl” refers to a radical of the formula —R_{b}—Rₑ where R_{b} is an alkynylene group as defined above and Rₑ is a heterocyclyl radical as defined above. Unless stated otherwise specifically in the specification, a heterocyclylalkynyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical one to thirteen carbon atoms and one to six heteroatoms selected from the group consisting of nitrogen, oxygen and sulfur, as the ring member. For purposes of this invention, the heteroaryl radical can be a monocyclic, bicyclic, tricyclic or tetracyclic ring system, which can include fused or bridged ring systems, wherein at least one ring containing a heteroatom ring member is aromatic. The nitrogen, carbon or sulfur atoms in the heteroaryl radical can be optionally oxidized and the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrazolopyridine, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}—R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}—R_{f} where R_{b} is an alkynylene chain as defined above and R_{f} is a heteroaryl radical as defined above. Unless stated otherwise specifically in the specification, a heteroarylalkynyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (e.g., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl, heteroarylalkyl, heteroarylalkenyl, heteroarylalkynyl, etc.) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

In addition, the aforementioned functional groups may, if a particular group permits, be further substituted with one or more additional functional groups or with one or more hydrocarbyl moieties, such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing”appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, substituted alkenyl, and substituted aryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

As used herein, the symbol

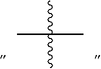

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

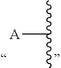

indicates that the chemical entity “A” is bonded to another chemical entity via the point of attachment bond. Furthermore, the specific point of attachment to the non-depicted chemical entity can be specified by inference. For example, the compound

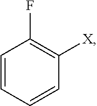

wherein X is

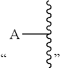

infers that the point of attachment bond is the bond by which X is depicted as being attached to the phenyl ring at the ortho position relative to fluorine.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

The term “ED50” is art-recognized. In certain embodiments, ED50 means the dose of a drug, which produces 50% of its maximum response or effect, or alternatively, the dose, which produces a pre-determined response in 50% of test subjects or preparations. The term “LD50” is art-recognized. In certain embodiments, LD50 means the dose of a drug, which is lethal in 50% of test subjects. The term “therapeutic index” is an art-recognized term, which refers to the therapeutic index of a drug, defined as LD50/ED50.

The terms “IC₅₀,” or “half maximal inhibitory concentration” is intended to refer to the concentration of a substance (e.g., a compound or a drug) that is required for 50% inhibition of a biological process, or component of a process, including a protein, subunit, organelle, ribonucleoprotein, etc.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

It will be noted that the structure of some of the compounds of the application include asymmetric (chiral) carbon or sulfur atoms. It is to be understood accordingly that the isomers arising from such asymmetry are included herein, unless indicated otherwise. Such isomers can be obtained in substantially pure form by classical separation techniques and by stereochemically controlled synthesis. The compounds of this application may exist in stereoisomeric form, therefore, can be produced as individual stereoisomers or as mixtures.

The term “isomerism” means compounds that have identical molecular formulae but that differ in the nature or the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images are termed “enantiomers”, or sometimes optical isomers. A carbon atom bonded to four nonidentical substituents is termed a “chiral center” whereas a sulfur bound to three or four different substitutents, e.g., sulfoxides or sulfinimides, is likewise termed a “chiral center”.

The term “chiral isomer” means a compound with at least one chiral center. It has two enantiomeric forms of opposite chirality and may exist either as an individual enantiomer or as a mixture of enantiomers. A mixture containing equal amounts of individual enantiomeric forms of opposite chirality is termed a “racemic mixture”. A compound that has more than one chiral center has 2n−1 enantiomeric pairs, where n is the number of chiral centers. Compounds with more than one chiral center may exist as either an individual diastereomer or as a mixture of diastereomers, termed a “diastereomeric mixture”. When one chiral center is present, a stereoisomer may be characterized by the absolute configuration (R or S) of that chiral center. Alternatively, when one or more chiral centers are present, a stereoisomer may be characterized as (+) or (−). Absolute configuration refers to the arrangement in space of the substituents attached to the chiral center. The substituents attached to the chiral center under consideration are ranked in accordance with the Sequence Rule of Cahn, Ingold and Prelog. (Cahn et al, Angew. Chem. Inter. Edit. 1966, 5, 385; errata 511; Cahn et al., Angew. Chem. 1966, 78, 413; Cahn and Ingold, J Chem. Soc. 1951 (London), 612; Cahn et al., Experientia 1956, 12, 81; Cahn, J., Chem. Educ. 1964, 41, 116).

The term “geometric Isomers” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules. Further, the structures and other compounds discussed in this application include all atropic isomers thereof.

The term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

The terms “crystal polymorphs” or “polymorphs” or “crystal forms” means crystal structures in which a compound (or salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The term “derivative” refers to compounds that have a common core structure, and are substituted with various groups as described herein.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include acyl sulfonimides, tetrazoles, sulfonates, and phosphonates. See, e.g., Patani and LaVoie, Chem. Rev. 96, 3147-3176 (1996).

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The term “pharmaceutical composition” refers to a formulation containing the disclosed compounds in a form suitable for administration to a subject. In a preferred embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed compound or salts thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, inhalational, and the like. Dosage forms for the topical or transdermal administration of a compound described herein includes powders, sprays, ointments, pastes, creams, lotions, gels, solutions, patches, nebulized compounds, and inhalants. In a preferred embodiment, the active compound is mixed under sterile conditions with a pharmaceutically acceptable carrier, and with any preservatives, buffers, or propellants that are required.

The term “flash dose” refers to compound formulations that are rapidly dispersing dosage forms.

The term “immediate release” is defined as a release of compound from a dosage form in a relatively brief period of time, generally up to about 60 minutes. The term “modified release” is defined to include delayed release, extended release, and pulsed release. The term “pulsed release” is defined as a series of releases of drug from a dosage form. The term “sustained release” or “extended release” is defined as continuous release of a compound from a dosage form over a prolonged period.

The compounds described herein can also be prepared as esters, for example pharmaceutically acceptable esters. For example, a carboxylic acid function group in a compound can be converted to its corresponding ester, e.g., a methyl, ethyl, or other ester. Also, an alcohol group in a compound can be converted to its corresponding ester, e.g., an acetate, propionate, or other ester.

The compounds described herein can also be prepared as prodrugs, for example pharmaceutically acceptable prodrugs. The terms “pro-drug” and “prodrug” are used interchangeably herein and refer to any compound, which releases an active parent drug in vivo. Since prodrugs are known to enhance numerous desirable qualities of pharmaceuticals (e.g., solubility, bioavailability, manufacturing, etc.) the compounds can be delivered in prodrug form. Thus, the compounds described herein are intended to cover prodrugs of the presently claimed compounds, methods of delivering the same and compositions containing the same. “Prodrugs” are intended to include any covalently bonded carriers that release an active parent drug in vivo when such prodrug is administered to a subject. Prodrugs are prepared by modifying functional groups present in the compound in such a way that the modifications are cleaved, either in routine manipulation or in vivo, to the parent compound. Prodrugs include compounds wherein a hydroxy, amino, sulfhydryl, carboxy, or carbonyl group is bonded to any group that may be cleaved in vivo to form a free hydroxyl, free amino, free sulfhydryl, free carboxy or free carbonyl group, respectively. Prodrugs can also include a precursor (forerunner) of a compound described herein that undergoes chemical conversion by metabolic processes before becoming an active or more active pharmacological agent or active compound described herein.

Examples of prodrugs include, but are not limited to, esters (e.g., acetate, dialkylaminoacetates, formates, phosphates, sulfates, and benzoate derivatives) and carbamates (e.g., N,N-dimethylaminocarbonyl) of hydroxy functional groups, ester groups (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds, and the like, as well as sulfides that are oxidized to form sulfoxides or sulfones.

The term “protecting group” refers to a grouping of atoms that when attached to a reactive group in a molecule masks, reduces or prevents that reactivity. Examples of protecting groups can be found in Green and Wuts, Protective Groups in Organic Chemistry, (Wiley, 2.sup.nd ed. 1991); Harrison and Harrison et al., Compendium of Synthetic Organic Methods, Vols. 1-8 (John Wiley and Sons, 1971-1996); and Kocienski, Protecting Groups, (Verlag, 3^{rd} ed. 2003).

The term “amine protecting group” is intended to mean a functional group that converts an amine, amide, or other nitrogen-containing moiety into a different chemical group that is substantially inert to the conditions of a particular chemical reaction. Amine protecting groups are preferably removed easily and selectively in good yield under conditions that do not affect other functional groups of the molecule. Examples of amine protecting groups include, but are not limited to, formyl, acetyl, benzyl, t-butyldimethylsilyl, t-butyldiphenylsilyl, t-butyloxycarbonyl (Boc), p-methoxybenzyl, methoxymethyl, tosyl, trifluoroacetyl, trimethylsilyl (TMS), fluorenyl-methyloxycarbonyl, 2-trimethylsilyl-ethyoxycarbonyl, 1-methyl-1-(4-biphenylyl) ethoxycarbonyl, allyloxycarbonyl, benzyloxycarbonyl (CBZ), 2-trimethylsilyl-ethanesulfonyl (SES), trityl and substituted trityl groups, 9-fluorenylmethyloxycarbonyl (FMOC), nitro-veratryloxycarbonyl (NVOC), and the like. Those of skill in the art can identify other suitable amine protecting groups.

Representative hydroxy protecting groups include those where the hydroxy group is either acylated or alkylated such as benzyl, and trityl ethers as well as alkyl ethers, tetrahydropyranyl ethers, trialkylsilyl ethers and allyl ethers.

The term “analogue” refers to a chemical compound that is structurally similar to another but differs slightly in composition (as in the replacement of one atom by an atom of a different element or in the presence of a particular functional group, or the replacement of one functional group by another functional group). Thus, an analogue is a compound that is similar or comparable in function and appearance, but not in structure or origin to the reference compound.

The terms “stable compound” and “stable structure” are meant to indicate a compound that is sufficiently robust to survive isolation, and as appropriate, purification from a reaction mixture, and formulation into an efficacious therapeutic agent.

The terms “free compound” is used herein to describe a compound in the unbound state.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “small molecule” is an art-recognized term. In certain embodiments, this term refers to a molecule, which has a molecular weight of less than about 2000 amu, or less than about 1000 amu, and even less than about 500 amu.

All percentages and ratios used herein, unless otherwise indicated, are by weight.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

It should be noted that throughout the application that alternatives are written in Markush groups, for example, each substituent position in a compound that contains more than one possible substituent. It is specifically contemplated that each member of the Markush group should be considered separately, thereby comprising another embodiment, and the Markush group is not to be read as a single unit.

Description of compounds described herein are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A “boronic acid”, as used herein, means a substituent with the structure —B(OH)₂. The term “boronic ester” is used according to its plain ordinary meaning and refers to a compound formed between a boronic acid and one or more alcohols (e.g., boronic acid pinacol ester or boronic acid pinanediol ester).

The term “cephalosporin” is used according to its plain ordinary meaning and refers to compounds that are derived from or related to 7-aminocephalosporanic acid, including modifications wherein functional groups or sidechains of the core 7-aminocephalosporanic acid group are modified or removed. The term “cephems” refers to the group of antibiotics comprising the cephalosporins and cephamycins.

The term “penicillin” is used according to its plain ordinary meaning and refers to compounds that are derived from or related to 6-aminopenicillanic acid, including modifications wherein functional groups or sidechains of the core 6-aminopenicillanic acid group are modified or removed.

The term “carbapenem” is used according to its plain ordingary meaning and refers to a class of beta lactam containing antibiotics including imipenem, meropenem, ertapenem, doripenem, panipenem, betamipron, biapenem, and tebipenem.

Cephalosporins are well known to be categorized into generations. When referring to a generation of cephalosporins herein, each generation is defined as commonly defined in the United States by medical practitioners or reference guides. “First generation cephalosporins” include Cefacetrile (cephacetrile), Cefadroxil (cefadroxyl; Duricef), Cephalexin (cephalexin; Keflex), Cefaloglycin (cephaloglycin), Cefalonium (cephalonium), Cefaloridine (cephaloradine), Cefalotin (cephalothin; Keflin), Cefapirin (cephapirin; Cefadryl), Cefatrizine, Cefazaflur, Cefazedone, Cefazolin (cephazolin; Ancef, Kefzol), Cefradine (cephradine; Velosef), Cefroxadine, Ceftezole. “Second generation cephalosporins” include Cefaclor (Ceclor, Distaclor, Keflor, Raniclor), Cefonicid (Monocid), Cefprozil (cefproxil; Cefzil), Cefuroxime (Zefu, Zinnat, Zinacef, Ceftin, Biofuroksym, Xorimax), Cefuzonam, Cefmetazole, Cefotetan, Cefoxitin. The following cephems are also sometimes grouped with second-generation cephalosporins: Carbacephems: loracarbef (Lorabid); Cephamycins: cefbuperazone, cefmetazole (Zefazone), cefminox, cefotetan (Cefotan), cefoxitin (Mefoxin). “Third generation cephalosporins” include Cefcapene, Cefdaloxime, Cefdinir (Zinir, Omnicef, Kefnir), Cefditoren, Cefetamet, Cefixime (Zifi, Suprax), Cefmenoxime, Cefodizime, Cefotaxime (Claforan), Cefovecin (Convenia), Cefpimizole, Cefpodoxime (Vantin, PECEF), Cefteram, Ceftibuten (Cedax), Ceftiofur, Ceftiolene, Ceftizoxime (Cefizox), Ceftriaxone (Rocephin), Cefoperazone (Cefobid), Ceftazidime (Fortum, Fortaz). The following cephems are also sometimes grouped with third-generation cephalosporins: Oxacephems: latamoxef (moxalactam). “Fourth generation cephalosporins” include Cefclidine, Cefepime (Maxipime), Cefluprenam, Cefoselis, Cefozopran, Cefpirome (Cefrom), Cefquinome, Oxacephems, flomoxef. “Fifth generation cephalosporins” include Ceftobiprole, Ceftaroline. Additional cephems include Cefaloram, Cefaparole, Cefcanel, Cefedrolor, Cefempidone, Cefetrizole, Cefivitril, Cefmatilen, Cefmepidium, Cefoxazole, Cefrotil, Cefsumide, Ceftioxide, Cefuracetime.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being, or improving the effectiveness of a method or compound administered to the patient for the purpose of treating the same disease (e.g., improving the effectiveness of an agent (i antibiotic, antimicrobial, antibacterial) administered for treatment of a disease). The treatment or amelioration of symptoms can be based on objective or subjective parameters; including the results of a physical examination, neuropsychiatric exams, and/or a psychiatric evaluation. For example, the certain methods presented herein successfully treat infectious diseases by, without being limited by mechanism, decreasing the growth, survival, and/or replication of infectious agents (e.g., bacteria), decreasing the growth, survival, and/or replication of infectious agents (e.g., bacteria) resistant to one or more antibiotics (e.g., penicillins, cephalosporins, β-lactam containing compounds, β-lactam containing antibiotics/β-lactam antibiotics), sensitizing infectious agents to one or more antibiotics (e.g., β-lactam containing compounds or β-lactam containing antibiotics/β-lactam antibiotics), inhibiting bacterial cell wall synthesis, inhibiting β-lactamase activity. The term “treating,” and conjugations thereof, include prevention of an injury, pathology, condition, or disease.

The terms “antibiotic” and “antibacterial” are used according to their plain ordinary meaning, as would be understood by a medical professional, and refer to compounds that kill or slow the growth of bacteria. The term “antimicrobial” is used according to its plain ordinary meaning, as would be understood by a medical professional, and refers to compounds that kill or slow the growth of microbes (e.g., bacteria, viruses, fungi, certain parasites). An “effective amount” is an amount sufficient to accomplish a stated purpose (e.g., achieve the effect for which it is administered, treat a disease, reduce enzyme activity, reduce one or more symptoms of a disease or condition, reduce the level of β-lactamase activity, sensitize an infectious agent to a second therapeutic agent (e.g., antibiotic, β-lactam containing compound, β-lactam containing antibiotic/β-lactam antibiotic, penicillin, cephalosporin). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist or compound required to decrease the activity of an enzyme (e.g., β-lactamase) relative to the absence of the antagonist or compound. A “function disrupting amount,” as used herein, refers to the amount of antagonist or compound required to disrupt the function of an enzyme or protein (e.g., β-lactamase) relative to the absence of the antagonist or compound. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g., chemical compounds including biomolecules, proteins, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a protein (e.g., β-lactamase). In some embodiments, the protein may be an enzyme. In some embodiments, the enzyme may be a β-lactamase. In some embodiments, the β-lactamase may be a class A β-lactamase). In some embodiments, the β-lactamase may be a class B β-lactamase). In some embodiments, the β-lactamase may be a class C β-lactamase). In some embodiments, the β-lactamase may be a class D β-lactamase). In some embodiments, contacting includes allowing a compound described herein to interact with a protein or enzyme that promotes the hydrolysis of β-lactam containing compounds. In some embodiments, contacting includes allowing a compound described herein to interact with an enzyme active site through specific amino acids (e.g., side chain atom or backbone atom). In some embodiments, contacting includes allowing a compound described herein to interact with an enzyme active site through specific amino acids identified through an x-ray crystal structure of the enzyme, or a similar or homologous enzyme, interacting with a substrate or compound described herein.

The terms “β-lactamase” or “β-lactamase” or “β-lactamase enzyme”, are used interchangeably and according to their plain ordinary meaning and refer to an enzyme or enzymes that catalyze the hydrolysis of β-lactam rings. β-lactamase enzymes include those enzymes identified by the Enzyme Commission number EC 3.5.2.6. β-lactamases include the enzymes identified by the UniProt numbers P05193, AMPC_CITFR; P00811, AMPC_ECOLI; P05364, AMPC_ENTCL; Q48743, AMPC_LYSLA; P94958, AMPC_MORMO; O69773, AMPC_PROST; P24735, AMPC_PSEAE; P85302, AMPC_PSEFL; O05465, AMPC_PSYIM; P18539, AMPC_SERMA; P45460, AMPC_YEREN; Q9S424, BLA13_KLEPN; P67920, BLA1_ACTPL; Q44056, BLA1_AERHY; P10424, BLA1_BACCE; P28018, BLA1_BACMY; POAD63, BLA1_ECOLX; P18251, BLA1_ENTCL; P67918, BLA1_HAEIF; POAD64, BLA1_KLEPN; Q59514, BLA1_MORCA; P67919, BLA1_PASHA; P52700, BLA1_STEMA; Q03680, BLA1_STRCI; Q9S169, BLA24_ECOLX; Q9AHN9, BLA29_KLEPN; P10425, BLA2_BAC17; P04190, BLA2_BACCE; POA9Z7, BLA2_ECOLX; POA9Z8, BLA2_KLEPN; POA9Z9, BLA2_KLEPO; POAA00, BLA2_SALTY; P96465, BLA2_STEMA; P14560, BLA2_STRCI; Q93LM8, BLA34_ECOLX; P06548, BLA3_BACCE; P30896, BLA3_KLEPN; Q848S4, BLA46 KLEOX; P37323, BLA4_KLEPN; POA3M1, BLA5 KLEPN; POA3M2, BLA5_PSEAE; P96348, BLA6_KLEPN; O08337, BLA8_ECOLX; O08498, BLAB1_FLAME; Q9RB01, BLAB2_FLAME; Q9K303, BLAB3_FLAME; Q9XBN7, BLAB4_FLAME; Q9KJA9, BLAB5_FLAME; Q9KJB0, BLAB6_FLAME; Q9KJA8, BLAB7_FLAME; Q9KJA7, BLAB8 FLAME; P26918, BLAB_AERHY; P14488, BLAB_BACCE; P25910, BLAB_BACFR; P52664, BLAB_PROVU; P52699, BLAB_SERMA; Q44674, BLAC_BACAM; P00809, BLAC_BACCE; P00808, BLAC_BACLI; P39824, BLAC_BACSU; Q45726, BLAC_BACTU; P30898, BLAC_BACUN; P30899, BLAC_BACVU; P22390, BLAC_CITDI; P05192, BLAC_KLEPN; POA517, BLAC_MYCBO; A5U493, BLAC_MYCTA; POC5C1, BLAC_MYCTU; Q9EZQ7, BLAC_NOCAS; Q5YXD6, BLAC_NOCFA; Q06316, BLAC_NOCLA; P30897, BLAC_PROMI; P80298, BLAC_PROVU; P14171, BLAC_RHOCA; P80545, BLAC_SERFO; P00807, BLAC_STAAU; P14559, BLAC_STRAL; P10509, BLAC_STRAU; P35391, BLAC_STRBA; Q06650, BLAC_STRCE; P35392, BLAC_STRFR; P81173, BLAC_STRGR; P35393, BLAC_STRLA; Q01166, BLAC_YEREN; Q59517, BLAF_MYCFO; C7C422, BLAN1_KLEPN; P52663, BLAN_ENTCL; P52682, BLAN_SERMA; P62593, BLAT_ECOLX; Q48406, BLAT_KLEOX; P62594, BLAT_SALTI; P28585, BLC1_ECOLX; P74841, BLC2_SALTY; P37322, BLC3_PSEAE; Q51355, BLC4_PSEAE; O33807, BLC4_SALTY; O65975, BLC5_SALTY; O65976, BLC6_SALTY; P81781, BLC6_VIBCH; P37321, BLEI_PSEAE; Q848S6, BLKPC_KLEOX; Q9F663, BLKPC_KLEPN; Q00983, BLL1_PSEAE; P14489, BLO10_PSEAE; Q06778, BLO11_PSEAE; Q51574, BLO15_PSEAE; O07293, BLO18_PSEAE; Q9R976, BLO19_PSEAE; P13661, BLO1_ECOLX; P22391, BLO1_KLEOX; O84955, BLO20_PSEAE; POA1V9, BLO2 ECOLX; P23954, BLO2_KLEOX; POA1V8, BLO2_SALTY; Q51429, BLO3_PSEAE; Q00982, BLOS_PSEAE; P35695, BLO7_ECOLX; POA3M4, BLO9_ENTAE; POA3M3, BLO9_KLEPN; Q03170, BLP1_PSEAE; P16897, BLP4_PSEAE; Q47066, BLT1_ECOLX; O69395, BLT2_ECOLX; POAEB2, DACA_ECOLI; Q9ZMM1, HCPA_HELPJ; O25001, HCPA_HELPY; O25103, HCPB_HELPY; Q9ZKB5, HCPC_HELPJ; O25728, HCPC_HELPY; Q9ZMSO, HCPD_HELPJ; O24968, HCPD_HELPY; Q9ZMJ9, HCPE_HELPJ; O25021, HCPE_HELPY; Q02940, PENA_BURM1; or P54427, YBXI_BACSU.

The term “inhibition”, “inhibit”, “inhibiting” and the like in reference to a protein-inhibitor (e.g., compound) interaction means negatively affecting (e.g., decreasing) the activity or function of the protein (e.g., decreasing the hydrolysis of β-lactam containing antibiotics) relative to the activity or function of the protein in the absence of the inhibitor (e.g., compound). In some embodiments inhibition refers to reduction of a disease or symptoms of disease. In some embodiments, inhibition refers to a reduction in the presence of a disease-related agent (e.g., an infectious agent, infectious agent resistant to one or more antibiotics, bacterium, bacterium resistant to one or more antibiotics). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly, an “inhibitor” is a compound that inhibits bacterial survival, growth, or replication, e.g., by binding, partially or totally blocking, decreasing, preventing, delaying, inactivating, desensitizing, or down-regulating signal transduction or enzymatic activity (e.g., activity responsible for hydrolyzing β-lactam containing compounds).

The term “modulator” refers to a composition that increases or decreases the level of a target molecule or the function of a target molecule (e.g., a target may be a β-lactamase and the function in a disease state of a β-lactamase, may be to hydrolyze therapeutic compounds such as β-lactam containing antibiotics). In some embodiments, a β-lactamase modulator is a compound that reduces the activity of a β-lactamase. A β-lactamase modulator may reduce an enzyme activity that results in a reduction of the amount of β-lactamase activity and reduces the amount of hydrolyzed β-lactam containing compounds or hydrolyzed β-lactam containing antibiotics produced by β-lactamases. In some embodiments, a β-lactamase modulator is a compound that reduces the severity of one or more symptoms of an infectious disease.

“Patient” or “subject in need thereof” refers to a living organism suffering from or prone to a condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refers to a state of being or health status of a patient or subject capable of being treated with the compounds or methods provided herein. In some embodiments, the disease is a disease related to (e.g., caused by) an infectious agent (e.g., bacteria) Examples of diseases, disorders, or conditions include, but are not limited to, infectious diseases, bacterial infectious diseases, nosocomial infections, nosocomial bacterial infections, ventilator associated pneumonias, bacterial blood stream infections, Cutaneous anthrax, Pulmonary anthrax, Gastrointestinal anthrax, Whooping cough, bacterial pneumonia, Lyme disease, Brucellosis, Acute enteritis, Community-acquired respiratory infection, Nongonococcal urethritis (NGU), Lymphogranuloma venereum (LGV), Trachoma, Inclusion conjunctivitis of the newborn (ICN), Psittacosis, Botulism, Pseudomembranous colitis, Gas gangrene, Acute food poisoning, Anaerobic cellulitis, Tetanus, Diphtheria, Nosocomial infections, Urinary tract infections (UTI), Diarrhea, Meningitis in infants, Traveler's diarrhea, Diarrhea in infants, Hemorrhagic colitis, Hemolytic-uremic syndrome, Tularemia, Bacterial meningitis, Upper respiratory tract infections, Pneumonia, bronchitis, Peptic ulcer, gastric carcinoma, gastric B-cell lymphoma, Legionnaire's Disease, Pontiac fever, Leptospirosis, Listeriosis, Leprosy (Hansen's disease), Tuberculosis, Mycoplasma pneumonia, Gonorrhea, Ophthalmia neonatorum, Septic arthritis, Meningococcal disease, Waterhouse-Friderichsen syndrome, Pseudomonas infection, Bacteremia, endocarditis, Rocky mountain spotted fever, Typhoid fever type salmonellosis (dysentery, colitis), Salmonellosis, gastroenteritis, enterocolitis, Bacillary dysentery/Shigellosis, Coagulase-positive staphylococcal infections, Impetigo, Acute infective endocarditis, Septicemia, Necrotizing pneumonia, Toxinoses, Toxic shock syndrome, Staphylococcal food poisoning, Cystitis, Meningitis, septicemia, Endometritis, Opportunistic infections, Acute bacterial pneumonia, Otitis media, sinusitis, Streptococcal pharyngitis, Scarlet fever, Rheumatic fever, erysipelas, Puerperal fever, Necrotizing fasciitis, Syphilis, Congenital syphilis, Cholera, Plague, Bubonic plague, Pneumonic plague, sepsis, Iraq war infection caused by Acinetobacter baumannii (i.e. Iraq war-related Acinetobacter baumannii infection), skin diseases or conditions, acne, acne vulgaris, keratosis pilaris, acne rosacea, harlequin ichthyosis, xeroderma pigmentosum, keratoses, eczema, rosacea, necrotizing fasciitis, tuberculosis, hospital-acquired pneumonia, gastroenteritis, or bacteremia.

The term “infectious disease” refers to a disease or condition related to the presence of an organism (the agent or infectious agent) within or contacting the subject or patient. Examples include a bacterium, fungus, virus, or other microorganism. A “bacterial infectious disease” is an infectious disease wherein the organism is a bacterium. A “viral infectious disease” is an infectious disease wherein the organism is a virus. An “antibiotic resistant bacterial infectious disease” is an infectious disease wherein the organism is a bacterium resistant to one or more antibiotics effective in treating a disease caused by the non-antibiotic resistant strains of the bacterium. A “penicillin resistant bacterial infectious disease” is an antibiotic resistant bacterial infectious disease wherein the disease is not treated as effectively by a penicillin or penicillin-related compounds as a similar disease caused by a bacterial strain that is not penicillin resistant. A “cephalosporin resistant bacterial infectious disease” is an antibiotic resistant bacterial infectious disease wherein the disease is not treated as effectively by a cephalosporin or cephalosporin-related compounds as a similar disease caused by a bacterial strain that is not cephalosporin resistant. A “β-lactam antibiotic resistant bacterial infectious disease” is an antibiotic resistant bacterial infectious disease wherein the disease is not treated as effectively by β-lactam containing antibiotics as a similar disease caused by a bacterial strain that is not β-lactam antibiotic resistant. Examples of infectious diseases that may be treated with a compound or method described herein include nosocomial infections, bacteremia, Cutaneous anthrax, Pulmonary anthrax, Gastrointestinal anthrax, Whooping cough, bacterial pneumonia, bacteremia, Lyme disease, Brucellosis, Acute enteritis, Community-acquired respiratory infection, Nongonococcal urethritis (NGU), Lymphogranuloma venereum (LGV), Trachoma, Inclusion conjunctivitis of the newborn (ICN), Psittacosis, Botulism, Pseudomembranous colitis, Gas gangrene, Acute food poisoning, Anaerobic cellulitis, Tetanus, Diphtheria, Nosocomial infections, Urinary tract infections (UTI), Diarrhea, Meningitis in infants, Traveler's diarrhea, Diarrhea in infants, Hemorrhagic colitis, Hemolytic-uremic syndrome, Tularemia, Bacterial meningitis, Upper respiratory tract infections, Pneumonia, bronchitis, Peptic ulcer, gastric carcinoma, gastric B-cell lymphoma, Legionnaire's Disease, Pontiac fever, Leptospirosis, Listeriosis, Leprosy (Hansen's disease), Tuberculosis, Mycoplasma pneumonia, Gonorrhea, Ophthalmia neonatorum, Septic arthritis, Meningococcal disease, Waterhouse-Friderichsen syndrome, Pseudomonas infection, Bacteremia, endocarditis, Rocky mountain spotted fever, Typhoid fever type salmonellosis (dysentery, colitis), Salmonellosis, gastroenteritis, enterocolitis, Bacillary dysentery/Shigellosis, Coagulase-positive staphylococcal infections: Impetigo, Acute infective endocarditis, Septicemia, Necrotizing pneumonia, Toxinoses, Toxic shock syndrome, Staphylococcal food poisoning, Cystitis, Meningitis, septicemia, Endometritis, Opportunistic infections, Acute bacterial pneumonia, Otitis media, sinusitis, Streptococcal pharyngitis, Scarlet fever, Rheumatic fever, erysipelas, Puerperal fever, Necrotizing fasciitis, Syphilis, Congenital syphilis, Cholera, Plague, Bubonic plague, Pneumonic plague, Iraq war infection caused by Acinetobacter baumannii (i.e., Iraq war-related Acinetobacter baumannii infection), necrotizing fasciitis, tuberculosis, hospital-acquired pneumonia, gastroenteritis, or sepsis.

“Infectious agent” refers to an organism that is associated with (in or contacting) patients with an infectious disease but not in patients without the infectious disease and wherein contacting a patient without the infectious disease with the organism results in the patient having the infectious disease. In some embodiments, the infectious agent associated with a disease that may be treated by the compounds and/or methods described herein is a bacterium. In some embodiments, the bacteria is of a genera selected from Stenotrophomonas, Clostridium, Acinetobacter, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, Klebsiella, Enterobacter, Citrobacter, or Yersinia. In some embodiments, the bacteria is selected from Stenotrophomonas maltophilia, Clostridium difficile, Bacillus anthracis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Enterotoxigenic Escherichia coli (ETEC), Enteropathogenic E. coli, E. coli O157: H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, Klebsiella pneumoniae, Enterobacter cloacae, Citrobacter freundii, Acinetobacter baumannii, or Yersinia pestis. In some embodiments, the bacteria are gram negative. In some embodiments, the bacteria are gram positive.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Embodiments described herein relate to compounds and methods of inhibiting β-lactamase activity as well as to compositions and methods of treating microbial or bacterial infections, such as β-lactam antibiotic resistant bacterial infections, in a subject in need thereof.

It was found that boronic acid compounds described herein can be used to inhibit and/or inactivate β-lactamase activity, and particularly, β-lactamase enzymatic function. The boronic acid β-lactamase inhibitors are therefore useful in the treatment of bacterial infections in subjects in need thereof alone or in combination with β-lactam antibiotics and/or with other non-β-lactam antibiotics.

In some embodiments, the boronic acid compound can have the structure of formula (I):

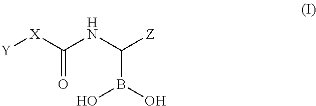

or a pharmaceutically acceptable salt, tautomer, or solvate thereof, wherein:
   X is alkylene or CNOR¹;
   Y is —W—R² or R²;
   Z is H, -alkylene-aryl, -alkylene-heterocyclyl, or -alkylene-heteroaryl, each of which is optionally substituted with one or more R³;
W is an S, N, O, S(O)ₘN, or alkylene optionally substituted with one or more halogen, alkyl, —COOH, —NH₂, or —OH;
R¹ is alkyl, -alkylene-OH optionally substituted with —OH, -alkylene-COOH optionally substituted with —OH, -alkylene-O-alkylene-OH, —C(O)-alkyl, —C(O)O-alkyl, or —C(O)O-alkylene-O—C(O)-alkyl;
R² is heterocyclyl or heteroaryl, each of which is optionally substituted with one or more R⁴;
each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), or —S(O₂)O⁻;
each R⁴ is independently halogen, alkyl, —COOH, —NH₂, or —OH;
each R⁵ is independently H or alkyl;
m is 0, 1, or 2; and
if X is CH₂, Y is —W—R².

In some embodiments, R¹ is C₁-C₆ alkyl, —(C₁-C₆ alkylene)-OH optionally substituted with —OH, —(C₁-C₆ alkylene)-COOH optionally substituted with —OH, —(C₁-C₆ alkylene)-O—(C₁-C₆ alkylene)-OH, —C(O)—(C₁-C₆ alkyl), —C(O)O—(C₁-C₆ alkyl), or —C(O)O—(C₁-C₆ alkylene)-O—C(O)—(C₁-C₆ alkyl).

In other embodiments, R¹ is C₁-C₆ alkyl or —(C₁-C₆ alkylene)-COOH optionally substituted with —OH.

In some embodiments, W is S, N, O, or S(O)ₘN; and m is 0, 1, or 2.

In some embodiments, R² is

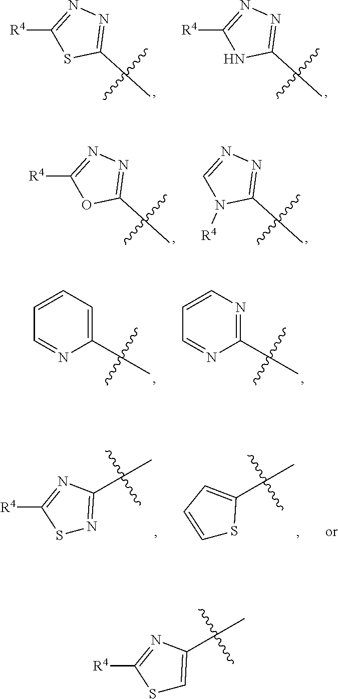

and R⁴ is halogen, alkyl, —COOH, —NH₂, —OH, or absent.

In some embodiments, Z is H, —CH₂-5- to 6-membered aryl, —CH₂-5- to 6-membered heterocyclyl, —CH₂-5- to 6-membered heteroaryl, each of which is optionally substituted with one or more R³.

In some embodiment, each R³ is independently —COOH,

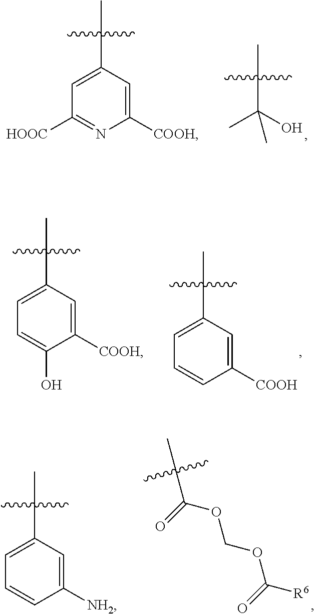

wherein R⁶ is alkyl, such as a C₁-C₆ alkyl, optionally substituted with one or more halogen, —COOH, —NH₂, or —OH.

In other embodiments, Z is H,

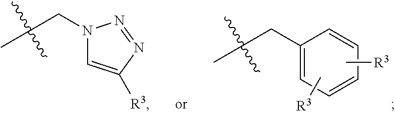

and each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻) C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), —S(O₂)O⁻ or absent; and each R⁵ is independently H or alkyl.

In some embodiments, the boronic acid compound can have the structure of formula (II):

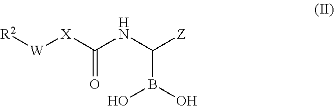

or a pharmaceutically acceptable salt, tautomer, or solvate thereof, wherein:
X is alkylene or CNOR¹;
Z is H, -alkylene-aryl, -alkylene-heterocyclyl, or -alkylene-heteroaryl, each of which is optionally substituted with one or more R³;
W is S, N, O, S(O)ₘN, or alkylene optionally substituted with one or more halogen, alkyl, —COOH, —NH₂, or —OH;
R¹ is alkyl, -alkylene-OH optionally substituted with —OH, -alkylene-COOH optionally substituted with —OH, -alkylene-O-alkylene-OH, —C(O)-alkyl, —C(O)O-alkyl, or —C(O)O-alkylene-O—C(O)-alkyl;
R² is heterocyclyl or heteroaryl, each of which is optionally substituted with one or more R⁴;
each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), or —S(O₂)O⁻;
each R⁴ is independently halogen, alkyl, —COOH, —NH₂, or —OH;
each R⁵ is independently H or alkyl; and
m is 0, 1, or 2.

In some embodiments, R¹ is C₁-C₆ alkyl, —(C₁-C₆ alkylene)-OH optionally substituted with —OH, —(C₁-C₆ alkylene)-COOH optionally substituted with —OH, —(C₁-C₆ alkylene)-O—(C₁-C₆ alkylene)-OH, —C(O)—(C₁-C₆ alkyl), —C(O)O—(C₁-C₆ alkyl), or —C(O)O—(C₁-C₆ alkylene)-O—C(O)—(C₁-C₆ alkyl).

In some embodiments, W is S, N, O, or S(O)ₘN; and m is 0, 1, or 2.

In other embodiments, R¹ is C₁-C₆ alkyl or —(C₁-C₆ alkylene)-COOH optionally substituted with —OH.

In some embodiments, R² is

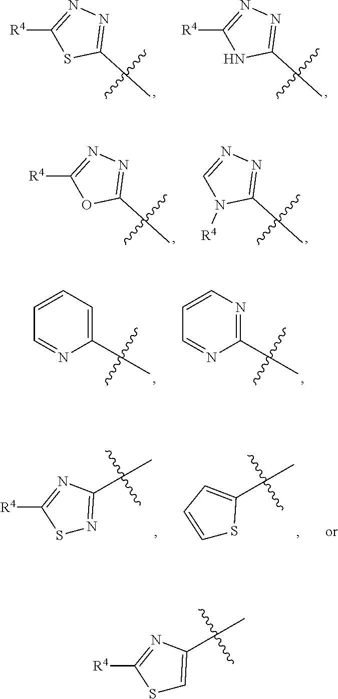

and R⁴ is halogen, alkyl, —COOH, —NH₂, —OH, or absent.

In some embodiments, Z is H, —CH₂-5- to 6-membered aryl, —CH₂-5- to 6-membered heterocyclyl, or —CH₂-5- to 6-membered heteroaryl, each of which is optionally substituted with one or more R³.

In some embodiment, each R³ is independently —COOH,

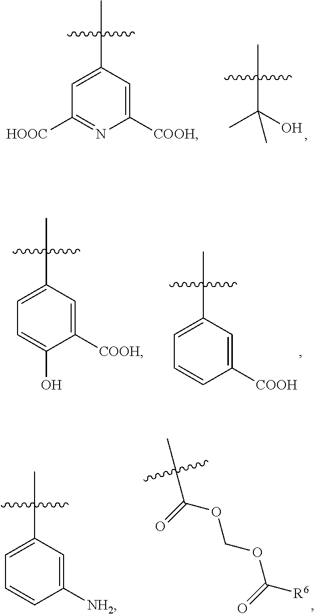

wherein R⁶ is alkyl, such as a C₁-C₆ alkyl, optionally substituted with one or more halogen, —COOH, —NH₂, or —OH.

In some embodiments, Z is H,

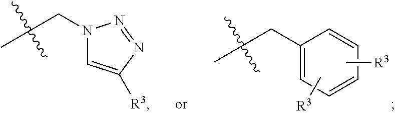

and each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻) C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), —S(O₂)O⁻, or absent; and each R⁵ is independently H or alkyl.

In other embodiments, boronic acid compound can have the structure of formula (III):

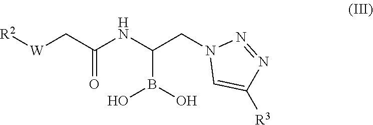

or a pharmaceutically acceptable salt, tautomer, or solvate thereof, wherein:
W is S, N, O, S(O)ₘN, or alkylene optionally substituted with one or more halogen, alkyl, —COOH, —NH₂, or —OH;
R² is heterocyclyl or heteroaryl, each of which is optionally substituted with one or more R⁴;
R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), —S(O₂)O⁻, or absent;
each R⁴ is independently halogen, alkyl, —COOH, —NH₂, or —OH; and
each R⁵ is independently H or alkyl.

In some embodiments, R² is

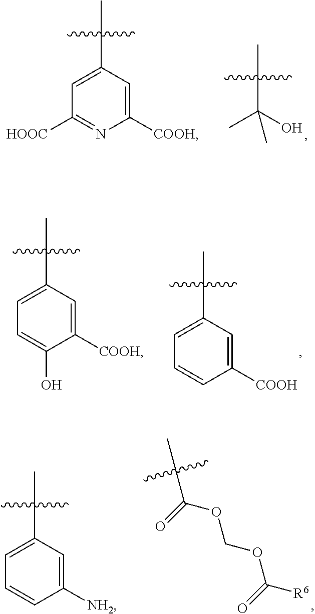

and R⁴ is halogen, alkyl, —COOH, —NH₂, —OH, or absent.

In some embodiments, W is S, N, O, or S(O)ₘN; and m is 0, 1, or 2.

In some embodiment, each R³ is independently —COOH,

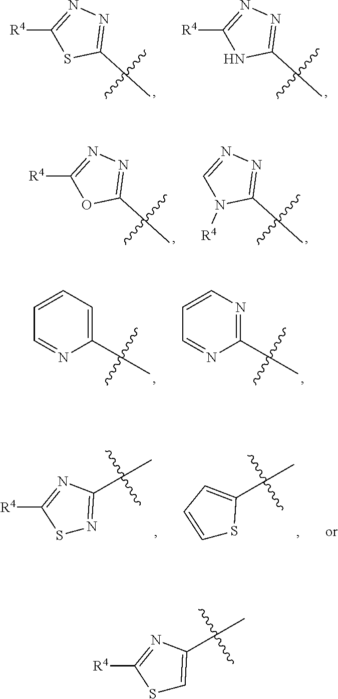

wherein R⁶ is alkyl, such as a C₁-C₆ alkyl, optionally substituted with one or more halogen, —COOH, —NH₂, or —OH.

In other embodiments, the boronic acid compound can have the structure of formula (IV):

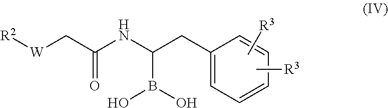

or a pharmaceutically acceptable salt, tautomer, or solvate thereof, wherein:
W is S, N, O, S(O)ₘN, or alkylene optionally substituted with one or more halogen, alkyl, —COOH, —NH₂, or —OH;
R² is heterocyclyl or heteroaryl, each of which is optionally substituted with one or more R⁴;
each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), —S(O₂)O⁻ or absent;
each R⁴ is independently halogen, alkyl, —COOH, —NH₂, or —OH; and
each R⁵ is independently H or alkyl.

In some embodiments, R² is:

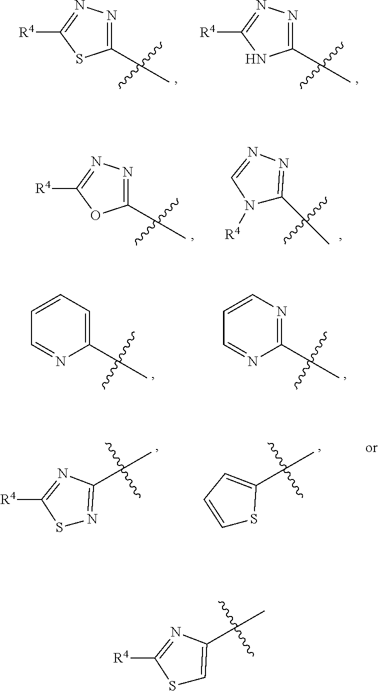

and R⁴ is halogen, alkyl, —COOH, —NH₂, —OH, or absent.

In some embodiments, W is S, N, O, or S(O)ₘN; and m is 0, 1, or 2.

In some embodiments, R³ is —COOH.

In still other embodiment, the boronic acid compound can have the structure of formula (V):

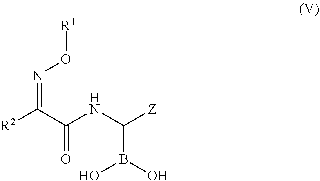

or a pharmaceutically acceptable salt, tautomer, or solvate thereof, wherein:
Z is H, -alkylene-aryl, -alkylene-heterocyclyl, or -alkylene-heteroaryl, each of which is optionally substituted with one or more R³;
R¹ is alkyl, -alkylene-OH optionally substituted with —OH, -alkylene-COOH optionally substituted with —OH, -alkylene-O-alkylene-OH, —C(O)-alkyl, —C(O)O-alkyl, or —C(O)O-alkylene-O—C(O)-alkyl;
R² is heterocyclyl or heteroaryl, each of which is optionally substituted with one or more R⁴;
each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), or —S(O₂)O⁻;
each R⁴ is independently halogen, alkyl, —COOH, —NH₂, or —OH; and
each R⁵ is independently H or alkyl.

In some embodiments, R¹ is C₁-C₆ alkyl, —(C₁-C₆ alkylene)-OH optionally substituted with —OH, —(C₁-C₆ alkylene)-COOH optionally substituted with —OH, —(C₁-C₆ alkylene)-O—(C₁-C₆ alkylene)-OH, —C(O)—(C₁-C₆ alkyl), —C(O)O—(C₁-C₆ alkyl), or —C(O)O—(C₁-C₆ alkylene)-O—C(O)—(C₁-C₆ alkyl).

In other embodiments, R¹ is C₁-C₆ alkyl or —(C₁-C₆ alkylene)-COOH optionally substituted with —OH.

In some embodiments, R² is

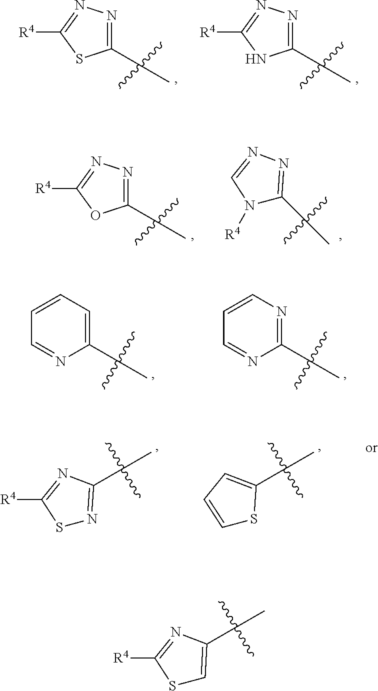

and R⁴ is halogen, alkyl, —COOH, —NH₂, —OH, or absent.

In some embodiments, Z is H, —CH₂-5- to 6-membered aryl, —CH₂-5- to 6-membered heterocyclyl, or —CH₂-5- to 6-membered heteroaryl, each of which is optionally substituted with one or more R³.

In some embodiment, each R³ is independently —COOH,

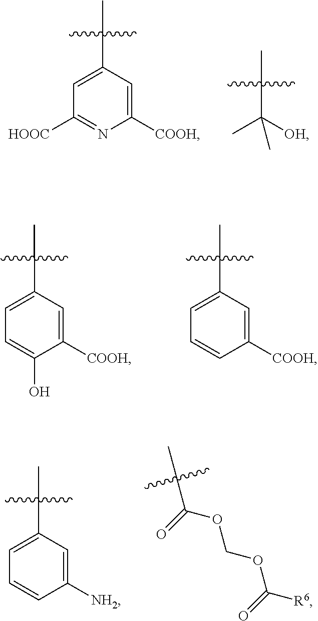

wherein R⁶ is alkyl, such as a C₁-C₆ alkyl, optionally substituted with one or more halogen, —COOH, —NH₂, or —OH.

In some embodiments, Z is H,

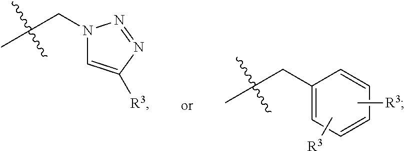

and
each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), or —S(O₂)O⁻ or absent; and each R⁵ is independently H or alkyl.

In some embodiments, the boronic acid compound does not have the structure:

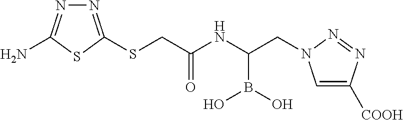 [SMILES: Nc1nnc(SCC(=O)NC(Cn2cc(C(=O)O)nn2)B(O)O)s1]

and a pharmaceutically acceptable salt, tautomer, and solvate thereof.

In some embodiments, the boronic acid compound has a structure of:

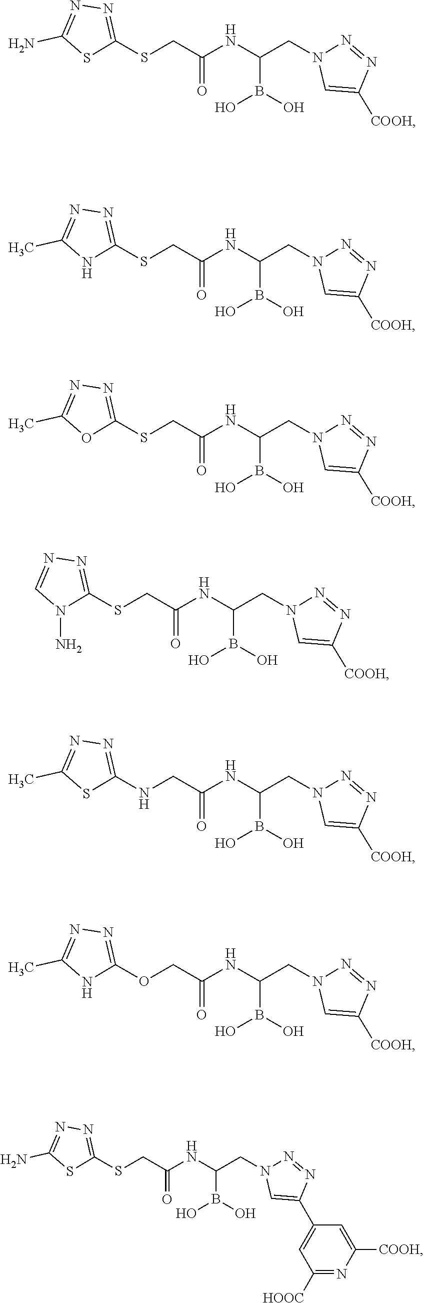

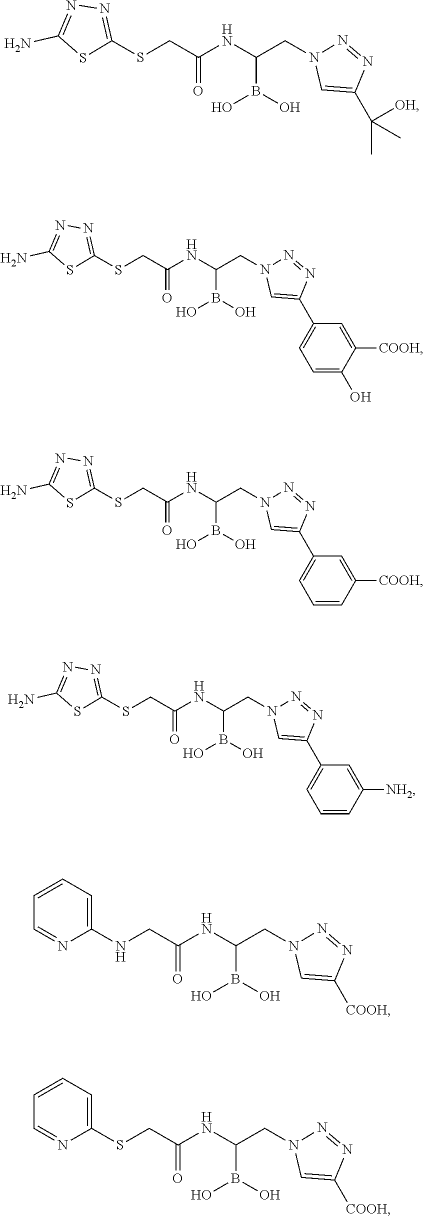 [SMILES: CC(C)(O)c1cn(CC(NC(=O)CSc2nnc(N)s2)B(O)O)nn1 ; Nc1nnc(SCC(=O)NC(Cn2cc(-c3ccc(O)c(C(=O)O)c3)nn2)B(O)O)s1 ; Nc1nnc(SCC(=O)NC(Cn2cc(-c3cccc(C(=O)O)c3)nn2)B(O)O)s1 ; Nc1cccc(-c2cn(CC(NC(=O)CSc3nnc(N)s3)B(O)O)nn2)c1 ; O=C(CNc1ccccn1)NC(Cn1cc(C(=O)O)nn1)B(O)O ; O=C(CSc1ccccn1)NC(Cn1cc(C(=O)O)nn1)B(O)O]

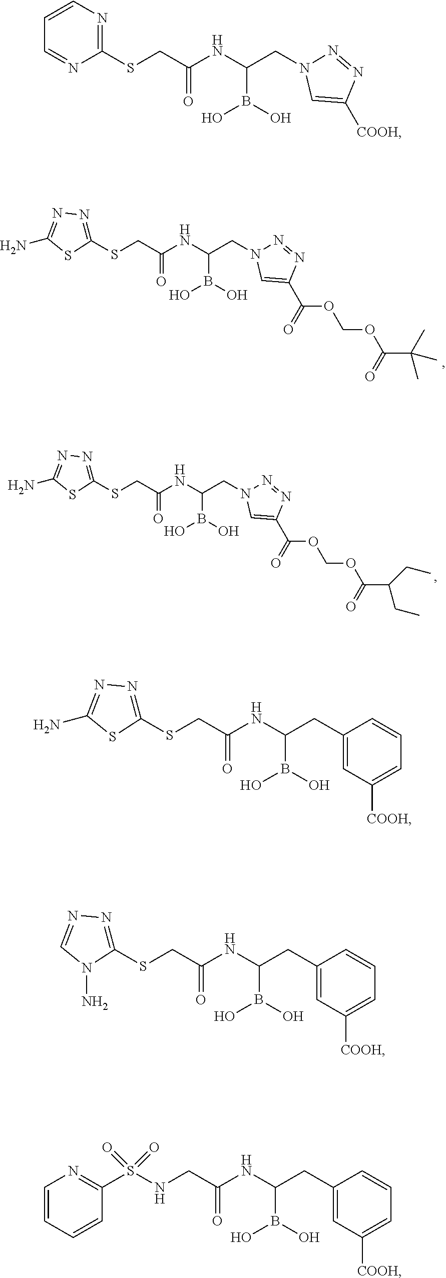 [SMILES: O=C(CSc1ncccn1)NC(Cn1cc(C(=O)O)nn1)B(O)O ; CC(C)(C)C(=O)OCOC(=O)c1cn(CC(NC(=O)CSc2nnc(N)s2)B(O)O)nn1 ; CCC(CC)C(=O)OCOC(=O)c1cn(CC(NC(=O)CSc2nnc(N)s2)B(O)O)nn1 ; Nc1nnc(SCC(=O)NC(Cc2cccc(C(=O)O)c2)B(O)O)s1 ; Nn1cnnc1SCC(=O)NC(Cc1cccc(C(=O)O)c1)B(O)O ; O=C(CNS(=O)(=O)c1ccccn1)NC(Cc1cccc(C(=O)O)c1)B(O)O]

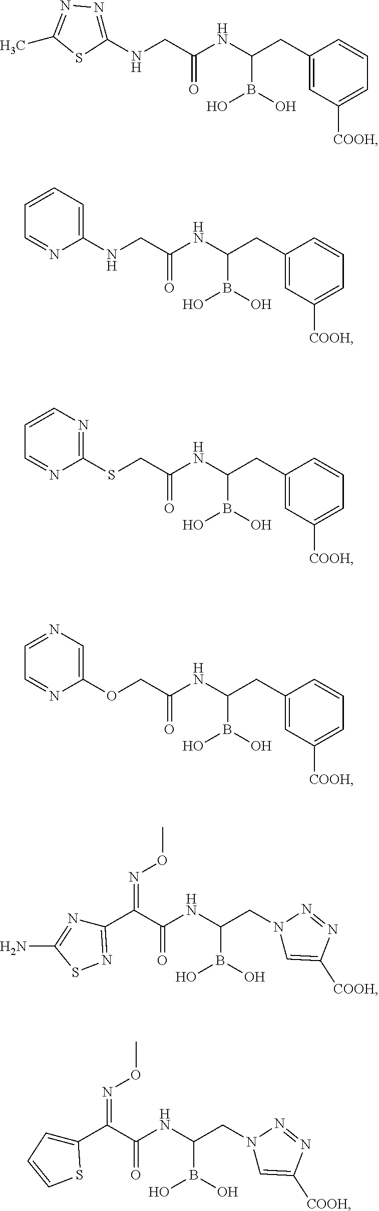 [SMILES: Cc1nnc(NCC(=O)NC(Cc2cccc(C(=O)O)c2)B(O)O)s1 ; O=C(CNc1ccccn1)NC(Cc1cccc(C(=O)O)c1)B(O)O ; O=C(CSc1ncccn1)NC(Cc1cccc(C(=O)O)c1)B(O)O ; O=C(COc1cnccn1)NC(Cc1cccc(C(=O)O)c1)B(O)O ; CO/N=C(\C(=O)NC(Cn1cc(C(=O)O)nn1)B(O)O)c1nsc(N)n1 ; CO/N=C(\C(=O)NC(Cn1cc(C(=O)O)nn1)B(O)O)c1cccs1]

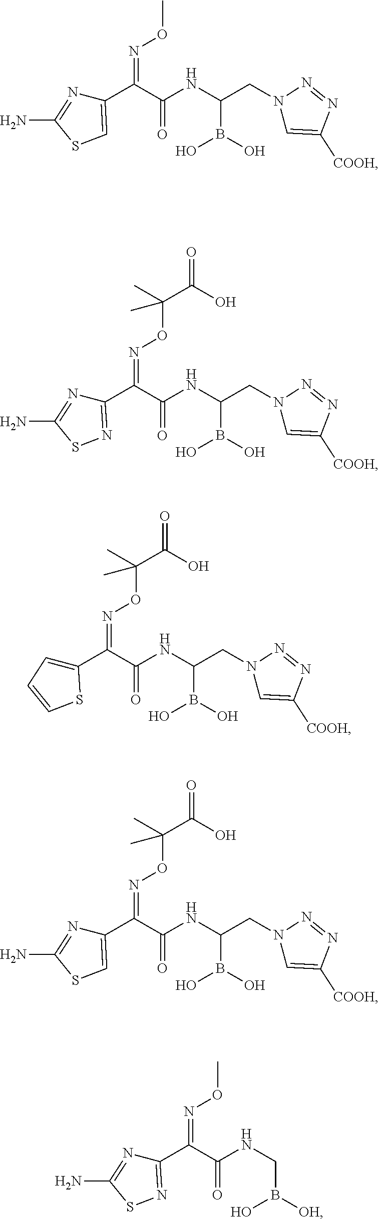 [SMILES: CO/N=C(\C(=O)NC(Cn1cc(C(=O)O)nn1)B(O)O)c1csc(N)n1 ; CC(C)(O/N=C(\C(=O)NC(Cn1cc(C(=O)O)nn1)B(O)O)c1nsc(N)n1)C(=O)O ; CC(C)(O/N=C(\C(=O)NC(Cn1cc(C(=O)O)nn1)B(O)O)c1cccs1)C(=O)O ; CC(C)(O/N=C(\C(=O)NC(Cn1cc(C(=O)O)nn1)B(O)O)c1csc(N)n1)C(=O)O ; CO/N=C(\C(=O)NCB(O)O)c1nsc(N)n1]

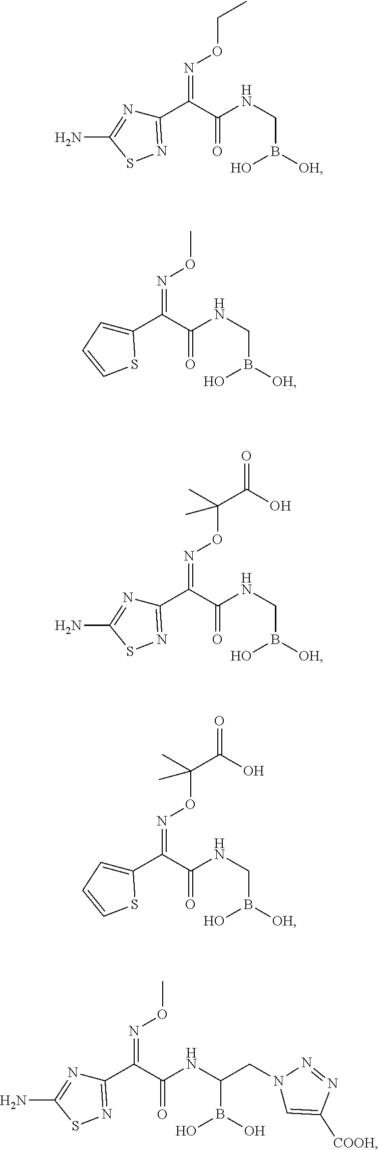 [SMILES: CCO/N=C(\C(=O)NCB(O)O)c1nsc(N)n1 ; CO/N=C(\C(=O)NCB(O)O)c1cccs1 ; CC(C)(O/N=C(\C(=O)NCB(O)O)c1nsc(N)n1)C(=O)O ; CC(C)(O/N=C(\C(=O)NCB(O)O)c1cccs1)C(=O)O ; CO/N=C(\C(=O)NC(Cn1cc(C(=O)O)nn1)B(O)O)c1nsc(N)n1]

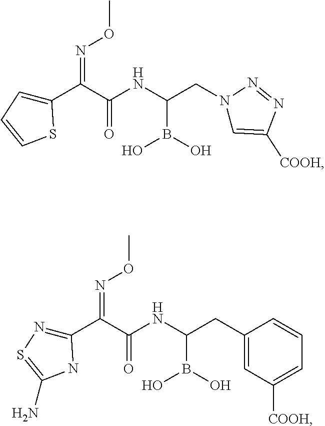

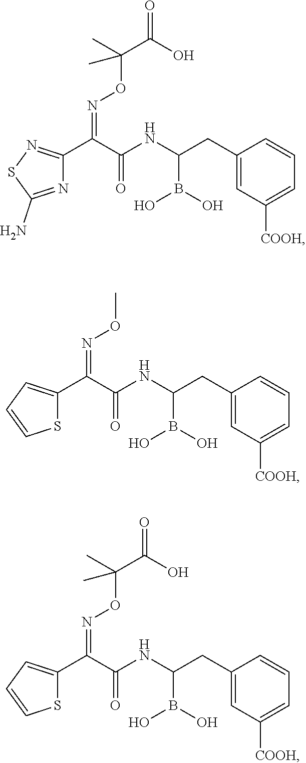 [SMILES: CC(C)(O/N=C(\C(=O)NC(Cc1cccc(C(=O)O)c1)B(O)O)c1nsc(N)n1)C(=O)O ; CO/N=C(\C(=O)NC(Cc1cccc(C(=O)O)c1)B(O)O)c1cccs1 ; CC(C)(O/N=C(\C(=O)NC(Cc1cccc(C(=O)O)c1)B(O)O)c1cccs1)C(=O)O]

or a pharmaceutically acceptable salt, tautomer, or solvate thereof.

In some embodiments of a compound described herein (e.g., Formula (I), (II), (III), (IV), or (V)), the compound inhibits the activity of a bacterial β-lactamase. In some embodiments, the bacterial β-lactamase is expressed by a bacterium selected from Stenotrophomonas maltophilia, Clostridium difficile, Bacillus anthracis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Enterotoxigenic Escherichia coli (ETEC), Enteropathogenic E. coli, E. coli O157: H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, Klebsiella pneumoniae, Enterobacter cloacae, Citrobacter freundii, Acinetobacter baumannii, or Yersinia pestis. In some embodiments, the bacterial β-lactamase is expressed by a bacterium that is gram negative. In some embodiments, the bacterial β-lactamase is expressed by a bacterium that is gram positive.

In some embodiments, the compounds described herein are not subject to degradation by β-lactamases or upregulation of these enzymes, unlike a classical β-lactam-based inhibitor.

In some embodiments, the boronic acid compounds retain substantial inhibition activity against β-lactamases. In other embodiments, they rescue antibiotic resistance when used in combination with third generation antibiotics in bacterial cell cultures.

The boronic acid compounds described herein can be used in a method of treating a disease in a patient in need of such treatment. The method can include administering a therapeutically effective amount of a compound as described herein (e.g., Formula (I), (II), (III), (IV), or (V)). In some embodiments, the disease is an infectious disease. In some embodiments, the disease is an infectious disease mediated by a bacterium. In some embodiments, the disease is an infectious disease caused by a bacterium. In some embodiments, the disease is a bacterial infectious disease. In further embodiments, the bacterium is resistant to an antibiotic. In further embodiments, the antibiotic is a β-lactam containing antibiotic. In some embodiments of the method of treating a disease, the bacterium is a gram negative bacterium. In some embodiments of the method of treating a disease, the bacterium is a gram positive bacterium. In some embodiments of the method of treating a disease in a patient in need of such treatment, the method includes administering a therapeutically effective amount of a compound as described herein (e.g., Formula (I), (II), (III), (IV), or (V)).

In further embodiments of the method of treating a disease, the genera of the bacterium is selected from Stenotrophomonas, Clostridium, Acinetobacter, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, Klebsiella, Enterobacter, Citrobacter, or Yersinia. In further embodiments of the method of treating a disease, the bacteria is selected from Stenotrophomonas maltophilia, Clostridium difficile, Bacillus anthracis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Enterotoxigenic Escherichia coli (ETEC), Enteropathogenic E. coli, E. coli O157: H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Klebsiella pneumoniae, Enterobacter cloacae, Citrobacter freundii, Vibrio cholerae, Acinetobacter baumannii, or Yersinia pestis. In further embodiments of the method of treating a disease, the bacteria is selected from a β-lactam antibiotic resistant strain of Stenotrophomonas maltophilia, Clostridium difficile, Bacillus anthracis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Enterotoxigenic Escherichia coli (ETEC), Enteropathogenic E. coli, E. coli O157: H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, Klebsiella pneumoniae, Enterobacter cloacae, Citrobacter freundii, Acinetobacter baumannii or Yersinia pestis. In some embodiments of the method of treating a disease, the disease is selected from Cutaneous anthrax, Pulmonary anthrax, Gastrointestinal anthrax, Whooping cough, bacterial pneumonia, Lyme disease, Brucellosis, Acute enteritis, Community-acquired respiratory infection, Nongonococcal urethritis (NGU), Lymphogranuloma venereum (LGV), Trachoma, Inclusion conjunctivitis of the newborn (ICN), Psittacosis, Botulism, Pseudomembranous colitis, Gas gangrene, Acute food poisoning, Anaerobic cellulitis, Tetanus, Diphtheria, Nosocomial infections, Urinary tract infections (UTI), Diarrhea, Meningitis in infants, Traveler's diarrhea, Diarrhea in infants, Hemorrhagic colitis, Hemolytic-uremic syndrome, Tularemia, Bacterial meningitis, Upper respiratory tract infections, Pneumonia, bronchitis, Peptic ulcer, gastric carcinoma, gastric B-cell lymphoma, Legionnaire's Disease, Pontiac fever, Leptospirosis, Listeriosis, Leprosy (Hansen's disease), Tuberculosis, Mycoplasma pneumonia, Gonorrhea, Ophthalmia neonatorum, Septic arthritis, Meningococcal disease, Waterhouse-Friderichsen syndrome, Pseudomonas infection, Bacteremia, endocarditis, Rocky mountain spotted fever, Typhoid fever type salmonellosis (dysentery, colitis), Salmonellosis, gastroenteritis, enterocolitis, Bacillary dysentery/Shigellosis, Coagulase-positive staphylococcal infections: Impetigo, Acute infective endocarditis, Septicemia, Necrotizing pneumonia, Toxinoses, Toxic shock syndrome, Staphylococcal food poisoning, Cystitis, Meningitis, septicemia, Endometritis, Opportunistic infections, Acute bacterial pneumonia, Otitis media, sinusitis, Streptococcal pharyngitis, Scarlet fever, Rheumatic fever, erysipelas, Puerperal fever, Necrotizing fasciitis, Syphilis, Congenital syphilis, Cholera, Plague, Bubonic plague, Pneumonic plague, Iraq war infection caused by Acinetobacter baumannii (i.e., Iraq war-related Acinetobacter baumannii infection), necrotizing fasciitis, tuberculosis, hospital-acquired pneumonia, gastroenteritis, nosocomial infection, bacteremia, or sepsis. In some embodiments of the method of treating a disease, the disease is a nosocomial infection. In some embodiments of the method of treating a disease, the disease is ventilator associated pneumonia. In some embodiments of the method of treating a disease, the disease is bacteremia.

In some embodiments of the method of treating a disease, a compound as described herein (e.g., Formula (I), (II), (III), (IV), or (V)), is co-administered with an antibiotic. In some embodiments, the antibiotic is a β-lactam containing antibiotic. In some embodiments, the antibiotic is a cephalosporin. In some embodiments, the cephalosporin is a first generation cephalosporin. In some embodiments, the cephalosporin is a second generation cephalosporin. In some embodiments, the cephalosporin is a third generation cephalosporin. In some embodiments, the cephalosporin is a fourth generation cephalosporin. In some embodiments, the cephalosporin is a fifth generation cephalosporin. In some embodiments, the cephalosporin is a sixth generation cephalosporin. In some embodiments, the antibiotic is a penicillin. In some embodiments, the antibiotic is a carbapenem.

In a fifth aspect, a method of inhibiting the growth of a bacterium in a patient is provided, the method includes administering a therapeutically effective amount of a compound as described herein (e.g., Formula (I), (II), (III), (IV), or (V)). In some embodiments of the method of inhibiting the growth of a bacterium in a patient, the bacterium is killed. In some embodiments of the method of inhibiting the growth of a bacterium in a patient, replication of the bacterium is slowed. In some embodiments of the method of inhibiting the growth of a bacterium in a patient, the bacterium is a gram negative bacterium. In some embodiments of the method of inhibiting the growth of a bacterium in a patient, the bacterium is a gram positive bacterium. In some embodiments of the method of inhibiting the growth of a bacterium in a patient, the method includes administering a therapeutically effective amount of a compound as described herein (e.g., Formula (I), (II), (III), (IV), or (V)).

In further embodiments of the method of inhibiting the growth of a bacterium in a patient, the genera of the bacterium is selected from Stenotrophomonas, Clostridium, Acinetobacter, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, Klebsiella, Enterobacter, Citrobacter, or Yersinia. In further embodiments of the method of inhibiting the growth of a bacterium in a patient, the bacteria is selected from Stenotrophomonas maltophilia, Clostridium difficile, Bacillus anthracis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Enterotoxigenic Escherichia coli (ETEC), Enteropathogenic E. coli, E. coli O157: H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, Klebsiella pneumoniae, Enterobacter cloacae, Citrobacter freundii, Acinetobacter baumannii, or Yersinia pestis. In further embodiments of the method of inhibiting the growth of a bacterium in a patient, the bacteria is selected from a β-lactam antibiotic resistant strain of Stenotrophomonas maltophilia, Clostridium difficile, Bacillus anthracis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Enterotoxigenic Escherichia coli (ETEC), Enteropathogenic E. coli, E. coli O157: H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, Klebsiella pneumoniae, Enterobacter cloacae, Citrobacter freundii, Acinetobacter baumannii, or Yersinia pestis.

In other embodiments, a method of inhibiting the hydrolysis of a β-lactam antibiotic by a bacterially expressed β-lactamase in a patient is provided. The method includes administering an effective amount of a compound as described herein (e.g., Formula (I), (II), (III), (IV), or (V)). In some embodiments of the method of inhibiting the hydrolysis of a β-lactam antibiotic by a bacterially expressed β-lactamase in a patient, the bacterium is a gram negative bacterium. In some embodiments of the method of inhibiting the hydrolysis of a β-lactam antibiotic by a bacterially expressed β-lactamase in a patient, the bacterium is a gram positive bacterium. In some embodiments of the method of inhibiting the hydrolysis of a β-lactam antibiotic by a bacterially expressed β-lactamase in a patient, the method includes administering an effective amount of a compound as described herein ((e.g., Formula (I), (II), (III), (IV), or (V)).

In further embodiments of the method of inhibiting the hydrolysis of a β-lactam antibiotic by a bacterially expressed β-lactamase in a patient, the genera of the bacterium is selected from Stenotrophomonas, Clostridium, Acinetobacter, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, Klebsiella, Enterobacter, Citrobacter, or Yersinia. In further embodiments of the method of inhibiting the hydrolysis of a β-lactam antibiotic by a bacterially expressed β-lactamase in a patient, the bacteria is selected from Stenotrophomonas maltophilia, Clostridium difficile, Bacillus anthracis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Enterotoxigenic Escherichia coli (ETEC), Enteropathogenic E. coli, E. coli O157: H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, Klebsiella pneumoniae, Enterobacter cloacae, Citrobacter freundii, Acinetobacter baumannii, or Yersinia pestis. In further embodiments of the method of inhibiting the hydrolysis of a β-lactam antibiotic by a bacterially expressed β-lactamase in a patient, the bacteria is selected from a β-lactam antibiotic resistant strain of Stenotrophomonas maltophilia, Clostridium difficile, Bacillus anthracis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Enterotoxigenic Escherichia coli (ETEC), Enteropathogenic E. coli, E. coli O157: H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, Klebsiella pneumoniae, Enterobacter cloacae, Citrobacter freundii, Acinetobacter baumannii, or Yersinia pestis. In some embodiments of the method of inhibiting the hydrolysis of a β-lactam antibiotic by a bacterially expressed β-lactamase in a patient, the β-lactamase is a Class A β-lactamase. In some embodiments of the method of inhibiting the hydrolysis of a β-lactam antibiotic by a bacterially expressed β-lactamase in a patient, the β-lactamase is a Class B β-lactamase. In some embodiments of the method of inhibiting the hydrolysis of a β-lactam antibiotic by a bacterially expressed β-lactamase in a patient, the β-lactamase is a Class C β-lactamase. In some embodiments of the method of inhibiting the hydrolysis of a β-lactam antibiotic by a bacterially expressed β-lactamase in a patient, the β-lactamase is a Class D β-lactamase.

In further embodiments, a method of reducing the therapeutically effective amount of an antibiotic necessary to treat a patient in need of such treatment is provided, the method includes administering an effective amount of a compound as described herein (e.g., Formula (I), (II), (III), (IV), or (V)). In some embodiments, the antibiotic is a penicillin. In some embodiments, the antibiotic is a cephalosporin. In some embodiments, the antibiotic is a cephamycin. In some embodiments, the antibiotic is a first-generation cephalosporin. In some embodiments, the antibiotic is a second-generation cephalosporin. In some embodiments, the antibiotic is a third-generation cephalosporin. In some embodiments, the antibiotic is a fourth-generation cephalosporin. In some embodiments, the antibiotic is a fifth-generation cephalosporin. In some embodiments, the antibiotic is a sixth-generation cephalosporin. In some embodiments of the method of reducing the therapeutically effective amount of an antibiotic necessary to treat a patient in need of such treatment, the method includes administering an effective amount of a compound as described herein (e.g., Formula (I), (II), (III), (IV), or (V)).

In some embodiments of a method of reducing the therapeutically effective amount of an antibiotic necessary to treat a patient in need of such treatment, the antibiotic is selected from Cefacetrile (cephacetrile), Cefadroxil (cefadroxyl; Duricef), Cephalexin (cephalexin; Keflex), Cefaloglycin (cephaloglycin), Cefalonium (cephalonium), Cefaloridine (cephaloradine), Cefalotin (cephalothin; Keflin), Cefapirin (cephapirin; Cefadryl), Cefatrizine, Cefazaflur, Cefazedone, Cefazolin (cephazolin; Ancef, Kefzol), Cefradine (cephradine; Velosef), Cefroxadine, Ceftezole, Cefaclor (Ceclor, Distaclor, Keflor, Raniclor), Cefonicid (Monocid), Cefprozil (cefproxil; Cefzil), Cefuroxime (Zefu, Zinnat, Zinacef, Ceftin, Biofuroksym, Xorimax), Cefuzonam, Cefmetazole, Cefotetan, Cefoxitin, Carbacephems, loracarbef (Lorabid); Cephamycins: cefbuperazone, cefmetazole (Zefazone), cefminox, cefotetan (Cefotan), cefoxitin (Mefoxin), Cefcapene, Cefdaloxime, Cefdinir (Zinir, Omnicef, Kefnir), Cefditoren, Cefetamet, Cefixime (Zifi, Suprax), Cefmenoxime, Cefodizime, Cefotaxime (Claforan), Cefovecin (Convenia), Cefpimizole, Cefpodoxime (Vantin, PECEF), Cefteram, Ceftibuten (Cedax), Ceftiofur, Ceftiolene, Ceftizoxime (Cefizox), Ceftriaxone (Rocephin), Cefoperazone (Cefobid), Ceftazidime (Fortum, Fortaz), Oxacephems: latamoxef (moxalactam), Cefclidine, Cefepime (Maxipime), Cefluprenam, Cefoselis, Cefozopran, Cefpirome (Cefrom), Cefquinome, Oxacephems, flomoxef, Ceftobiprole, Ceftaroline, Cefaloram, Cefaparole, Cefcanel, Cefedrolor, Cefempidone, Cefetrizole, Cefivitril, Cefmatilen, Cefmepidium, Cefoxazole, Cefrotil, Cefsumide, Ceftioxide, or Cefuracetime.

In some embodiments of a method of treating a disease in a patient, reducing the therapeutically effective amount of an antibiotic necessary to treat a patient in need of such treatment, inhibiting the hydrolysis of a β-lactam antibiotic by a bacterially expressed β-lactamase in a patient, or inhibiting the growth of a bacterium in a patient, as described herein, the method further includes administering a β-lactam antibiotic to the patient. In some embodiments, the β-lactam antibiotic is a penicillin. In some embodiments, the β-lactam antibiotic is a cephalosporin. In some embodiments, the β-lactam antibiotic is a cephamycin. In some embodiments, the β-lactam antibiotic is a first-generation cephalosporin. In some embodiments, the β-lactam antibiotic is a second-generation cephalosporin. In some embodiments, the β-lactam antibiotic is a third-generation cephalosporin. In some embodiments, the β-lactam antibiotic is a fourth-generation cephalosporin. In some embodiments, the β-lactam antibiotic is a fifth-generation cephalosporin. In some embodiments, the β-lactam antibiotic is a sixth-generation cephalosporin. In some embodiments, the antibiotic is selected from Cefacetrile (cephacetrile), Cefadroxil (cefadroxyl; Duricef), Cephalexin (cephalexin; Keflex), Cefaloglycin (cephaloglycin), Cefalonium (cephalonium), Cefaloridine (cephaloradine), Cefalotin (cephalothin; Keflin), Cefapirin (cephapirin; Cefadryl), Cefatrizine, Cefazaflur, Cefazedone, Cefazolin (cephazolin; Ancef, Kefzol), Cefradine (cephradine; Velosef), Cefroxadine, Ceftezole, Cefaclor (Ceclor, Distaclor, Keflor, Raniclor), Cefonicid (Monocid), Cefprozil (cefproxil; Cefzil), Cefuroxime (Zefu, Zinnat, Zinacef, Ceftin, Biofuroksym, Xorimax), Cefuzonam, Cefmetazole, Cefotetan, Cefoxitin, Carbacephems, loracarbef (Lorabid); Cephamycins: cefbuperazone, cefmetazole (Zefazone), cefminox, cefotetan (Cefotan), cefoxitin (Mefoxin), Cefcapene, Cefdaloxime, Cefdinir (Zinir, Omnicef, Kefnir), Cefditoren, Cefetamet, Cefixime (Zifi, Suprax), Cefmenoxime, Cefodizime, Cefotaxime (Claforan), Cefovecin (Convenia), Cefpimizole, Cefpodoxime (Vantin, PECEF), Cefteram, Ceftibuten (Cedax), Ceftiofur, Ceftiolene, Ceftizoxime (Cefizox), Ceftriaxone (Rocephin), Cefoperazone (Cefobid), Ceftazidime (Fortum, Fortaz), Oxacephems: latamoxef (moxalactam), Cefclidine, Cefepime (Maxipime), Cefluprenam, Cefoselis, Cefozopran, Cefpirome (Cefrom), Cefquinome, Oxacephems, flomoxef, Ceftobiprole, Ceftaroline, Cefaloram, Cefaparole, Cefcanel, Cefedrolor, Cefempidone, Cefetrizole, Cefivitril, Cefmatilen, Cefmepidium, Cefoxazole, Cefrotil, Cefsumide, Ceftioxide, or Cefuracetime.

In further embodiments, a kit is provided for treating a bacterial infectious disease including a compound as described herein (e.g., Formula (I), (II), (III), (IV), or (V)) and a β-lactam containing antibiotic. In some embodiments of the kit for treating a bacterial infectious disease, the β-lactam containing antibiotic is a penicillin. In some embodiments of the kit for treating a bacterial infectious disease, the β-lactam containing antibiotic is a cephalosporin. In some embodiments, the β-lactam antibiotic is selected from Cefacetrile (cephacetrile), Cefadroxil (cefadroxyl; Duricef), Cephalexin (cephalexin; Keflex), Cefaloglycin (cephaloglycin), Cefalonium (cephalonium), Cefaloridine (cephaloradine), Cefalotin (cephalothin; Keflin), Cefapirin (cephapirin; Cefadryl), Cefatrizine, Cefazaflur, Cefazedone, Cefazolin (cephazolin; Ancef, Kefzol), Cefradine (cephradine; Velosef), Cefroxadine, Ceftezole, Cefaclor (Ceclor, Distaclor, Keflor, Raniclor), Cefonicid (Monocid), Cefprozil (cefproxil; Cefzil), Cefuroxime (Zefu, Zinnat, Zinacef, Ceftin, Biofuroksym, Xorimax), Cefuzonam, Cefmetazole, Cefotetan, Cefoxitin, Carbacephems, loracarbef (Lorabid); Cephamycins: cefbuperazone, cefmetazole (Zefazone), cefminox, cefotetan (Cefotan), cefoxitin (Mefoxin), Cefcapene, Cefdaloxime, Cefdinir (Zinir, Omnicef, Kefnir), Cefditoren, Cefetamet, Cefixime (Zifi, Suprax), Cefmenoxime, Cefodizime, Cefotaxime (Claforan), Cefovecin (Convenia), Cefpimizole, Cefpodoxime (Vantin, PECEF), Cefteram, Ceftibuten (Cedax), Ceftiofur, Ceftiolene, Ceftizoxime (Cefizox), Ceftriaxone (Rocephin), Cefoperazone (Cefobid), Ceftazidime (Fortum, Fortaz), Oxacephems: latamoxef (moxalactam), Cefclidine, Cefepime (Maxipime), Cefluprenam, Cefoselis, Cefozopran, Cefpirome (Cefrom), Cefquinome, Oxacephems, flomoxef, Ceftobiprole, Ceftaroline. Additional cephems include Cefaloram, Cefaparole, Cefcanel, Cefedrolor, Cefempidone, Cefetrizole, Cefivitril, Cefmatilen, Cefmepidium, Cefoxazole, Cefrotil, Cefsumide, Ceftioxide, or Cefuracetime. In some embodiments of the kit for treating a bacterial infectious disease including a compound as described herein (e.g., Formula (I), (II), (III), (IV), or (V)) and a β-lactam containing antibiotic.

In other embodiments, a pharmaceutical composition is provided including a pharmaceutically acceptable excipient and a compound as described herein ((e.g., Formula (I), (II), (III), (IV), or (V)). In some embodiments, the pharmaceutical composition includes an antibiotic. In some embodiments, the antibiotic is a β-lactam containing antibiotic. In some embodiments, the antibiotic is a penicillin. In some embodiments, the antibiotic is a cephalosporin. In some embodiments, the antibiotic is a cephamycin. In some embodiments, the antibiotic is a first-generation cephalosporin. In some embodiments, the antibiotic is a second-generation cephalosporin. In some embodiments, the antibiotic is a third-generation cephalosporin. In some embodiments, the antibiotic is a fourth-generation cephalosporin. In some embodiments, the antibiotic is a fifth-generation cephalosporin. In some embodiments, the antibiotic is a sixth-generation cephalosporin. In some embodiments of the pharmaceutical composition including a pharmaceutically acceptable excipient and a compound as described herein (e.g., Formula (I), (II), (III), (IV), or (V)).

In some embodiments of a pharmaceutical composition, the antibiotic is selected from Cefacetrile (cephacetrile), Cefadroxil (cefadroxyl; Duricef), Cephalexin (cephalexin; Keflex), Cefaloglycin (cephaloglycin), Cefalonium (cephalonium), Cefaloridine (cephaloradine), Cefalotin (cephalothin; Keflin), Cefapirin (cephapirin; Cefadryl), Cefatrizine, Cefazaflur, Cefazedone, Cefazolin (cephazolin; Ancef, Kefzol), Cefradine (cephradine; Velosef), Cefroxadine, Ceftezole, Cefaclor (Ceclor, Distaclor, Keflor, Raniclor), Cefonicid (Monocid), Cefprozil (cefproxil; Cefzil), Cefuroxime (Zefu, Zinnat, Zinacef, Ceftin, Biofuroksym, Xorimax), Cefuzonam, Cefmetazole, Cefotetan, Cefoxitin, Carbacephems, loracarbef (Lorabid); Cephamycins: cefbuperazone, cefmetazole (Zefazone), cefminox, cefotetan (Cefotan), cefoxitin (Mefoxin), Cefcapene, Cefdaloxime, Cefdinir (Zinir, Omnicef, Kefnir), Cefditoren, Cefetamet, Cefixime (Zifi, Suprax), Cefmenoxime, Cefodizime, Cefotaxime (Claforan), Cefovecin (Convenia), Cefpimizole, Cefpodoxime (Vantin, PECEF), Cefteram, Ceftibuten (Cedax), Ceftiofur, Ceftiolene, Ceftizoxime (Cefizox), Ceftriaxone (Rocephin), Cefoperazone (Cefobid), Ceftazidime (Fortum, Fortaz), Oxacephems: latamoxef (moxalactam), Cefclidine, Cefepime (Maxipime), Cefluprenam, Cefoselis, Cefozopran, Cefpirome (Cefrom), Cefquinome, Oxacephems, flomoxef, Ceftobiprole, Ceftaroline, Cefaloram, Cefaparole, Cefcanel, Cefedrolor, Cefempidone, Cefetrizole, Cefivitril, Cefmatilen, Cefmepidium, Cefoxazole, Cefrotil, Cefsumide, Ceftioxide, or Cefuracetime.

The pharmaceutical compositions include optical isomers, diastereomers, or pharmaceutically acceptable salts of the modulators disclosed herein. The compound included in the pharmaceutical composition may be covalently attached to a carrier moiety, as described above. Alternatively, the compound included in the pharmaceutical composition is not covalently linked to a carrier moiety.

The compounds described herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation).

The compounds described herein can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compounds of the present invention can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance, that may also act as diluents, flavoring agents, binders, preservatives, tablet disintegrating agents, or an encapsulating material.

In powders, the carrier is a finely divided solid in a mixture with the finely divided active component (e.g., a compound provided herein. In tablets, the active component is mixed with the carrier having the necessary binding properties in suitable proportions and compacted in the shape and size desired. The powders and tablets preferably contain from 5% to 70% of the active compound.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compounds of the invention can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60 and 80; Pluronic F-68, F-84 and P-103; cyclodextrin; polyoxyl 35 castor oil; or other agents known to those skilled in the art. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained by one skilled in the art.

The compositions described herein may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

Pharmaceutical compositions described herein may include compositions wherein the active ingredient is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g., β-lactamase, class A β-lactamase, class B β-lactamase, class C β-lactamase, or class D β-lactamase), and/or reducing, eliminating, or slowing the progression of disease symptoms. Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., infectious disease, bacterial infectious disease, antibiotic resistant bacterial infectious disease), kind of concurrent treatment, complications from the disease being treated or other health-related problems. Other therapeutic regimens or agents can be used in conjunction with the methods and compounds of described herein. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. In one embodiment, the dosage range is 0.001% to 10% w/v. In another embodiment, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g., Fingl et al., In: THE PHARMACOLOGICAL BASIS OF THERAPEUTICS, Ch. 1, p. 1, 1975. The exact formulation, route of administration and dosage can be chosen by the individual physician in view of the patient's condition and the particular method in which the compound is used.

The compositions described herein can be delivered transdermally, by a topical route, formulated as applicator sticks, solutions, suspensions, emulsions, gels, creams, ointments, pastes, jellies, paints, powders, and aerosols.

The compositions described herein can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The pharmaceutical compositions described herein can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the compositions described herein are useful for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions described herein can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds described herein can be used in combination with one another, with other active agents known to be useful in treating an infectious disease (antibiotic, penicillin, cephalosporin, β-lactam containing antibiotic), or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents (compound as described herein and a β-lactam containing antibiotic). In other embodiments, the active agents can be formulated separately. In another embodiment, the active and/or adjunctive agents may be linked or conjugated to one another.

In some embodiments, the boronic acid compounds and optionally the antibiotic can be administered prior to a bacterial infection, after infection but prior to the manifestation of symptoms of a disease of disorder associated with the infection, or after the manifestation of symptoms associated with the production of one or more bacterial virulence factors to prevent further bacterial multiplication and to prevent further production of virulence factors thereby hindering development of the disease or its progression.

In another aspect, the boronic acid compounds and β-lactam antibiotics can be used to treat bacteria on or associated with a medical device by contacting the device with the boronic acid compounds and β-lactam antibiotics.

A medical device according to the application can comprise any instrument, implement, machine, contrivance, implant, or other similar or related article, including a component or part, or accessory which is: recognized in the official U.S. National Formulary the U.S. Pharmacopoeia, or any supplement thereof; intended for use in the diagnosis of disease or other conditions, or in the cure, mitigation, treatment, or prevention of disease, in humans or in other animals; or, intended to affect the structure or any function of the body of humans or other animals, and which does not achieve any of its primary intended purposes through chemical action within or on the body of human or other animal, and which is not dependent upon being metabolized for the achievement of any of its primary intended purposes.

A medical device can include, for example, endovascular medical devices, such as intracoronary medical devices. Examples of intracoronary medical devices can include stents, drug delivery catheters, grafts, and drug delivery balloons utilized in the vasculature of a subject. Where the medical device comprises a stent, the stent may include peripheral stents, peripheral coronary stents, degradable coronary stents, non-degradable coronary stents, self-expanding stents, balloon-expanded stents, and esophageal stents. The medical device may also include arterio-venous grafts, by-pass grafts, penile implants, vascular implants and grafts, intravenous catheters, small diameter grafts, artificial lung catheters, electrophysiology catheters, bone pins, suture anchors, blood pressure and stent graft catheters, breast implants, benign prostatic hyperplasia and prostate cancer implants, bone repair/augmentation devices, breast implants, orthopedic joint implants, dental implants, implanted drug infusion tubes, oncological implants, pain management implants, neurological catheters, central venous access catheters, catheter cuff, vascular access catheters, urological catheters/implants, atherectomy catheters, clot extraction catheters, PTA catheters, PTCA catheters, stylets (vascular and non-vascular), drug infusion catheters, angiographic catheters, hemodialysis catheters, neurovascular balloon catheters, thoracic cavity suction drainage catheters, electrophysiology catheters, stroke therapy catheters, abscess drainage catheters, biliary drainage products, dialysis catheters, central venous access catheters, and parental feeding catheters.

The medical device may additionally include either arterial or venous pacemakers, vascular grafts, sphincter devices, urethral devices, bladder devices, renal devices, gastroenteral and anastomotic devices, vertebral disks, hemostatic barriers, clamps, surgical staples/sutures/screws/plates/wires/clips, glucose sensors, blood oxygenator tubing, blood oxygenator membranes, blood bags, birth control/IUDs and associated pregnancy control devices, cartilage repair devices, orthopedic fracture repairs, tissue scaffolds, CSF shunts, dental fracture repair devices, intravitreal drug delivery devices, nerve regeneration conduits, electrostimulation leads, spinal/orthopedic repair devices, wound dressings, embolic protection filters, abdominal aortic aneurysm grafts and devices, neuroaneurysm treatment coils, hemodialysis devices, uterine bleeding patches, anastomotic closures, aneurysm exclusion devices, neuropatches, vena cava filters, urinary dilators, endoscopic surgical and wound drainings, bandages, surgical tissue extractors, transition sheaths and dialators, coronary and peripheral guidewires, circulatory support systems, tympanostomy vent tubes, cerebro-spinal fluid shunts, defibrillator leads, percutaneous closure devices, drainage tubes, bronchial tubes, vascular coils, vascular protection devices, vascular intervention devices including vascular filters and distal support devices and emboli filter/entrapment aids, AV access grafts, surgical tampons, and cardiac valves.

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example

In this example, we describe the synthesis of compounds of general formula I that can be prepared according but not limited to the procedures shown schematically in Scheme 1 for compound MB076, in Scheme 2 for compound MB054, in Scheme 3 for compound PCF-013, in Scheme 4 for compound PCF-014, and in Scheme 5 for alkoxycarbonyloxymethyl derivatives.

### Synthesis of MB076 and Related Compounds

The general synthesis of α-amido-b-triazolylboronates MB076 and PCF038-043 is summarized in scheme 1. The starting (+)-pinanendiol (1R)-2-azido-1-[N,N-bis(trimethylsislyl)amino]-ethaneboronate 1 was synthesized according to the literature (J. Organomet. Chem. 2008, 693, 2258-2262). Removal of the TMS groups by stoichiometric methanol and subsequent reaction with cloroacetyl chloride at −30° C. afforded compound 2 in almost quantitative yield, which was used without any purification for the click reaction with tert-butylpropiolate (as described in J. Med. Chem. 2015, 545-5458) affording the b-triazolylboronate 3 (95% yield).

Compound 3 was the key intermediate, allowing different heterocycles to be inserted by nucleophilic substitution of the chlorine atom; reactions were performed in acetonitrile at room temperature affording the expected compounds 4 in 45 to 95% yields.

Each compound 4 was dissolved in Dry DCM and TFA (20 eq.) was added. The reaction mixture was stirred for 6 h and then evaporated to dryness and the crude carboxylic acid, without further purifications, was suspended in acetonitrile and hexane. Isobutylboronic acid (1 eq.) and aq. HCl (3 eq.) were added. The two phases were mixed with vigorous stirring for 30 min, whereupon the hexane phase containing pinanediol isobutylboronate was removed and replaced with fresh hexane. This procedure was repeated several times until TLC showed no pinanediol isobutylboronate in the hexane phase. The acetonitrile phase was concentrated in vacuo affording the the free boronic acids MB076 and PCF038-043. (70%-85% yield).

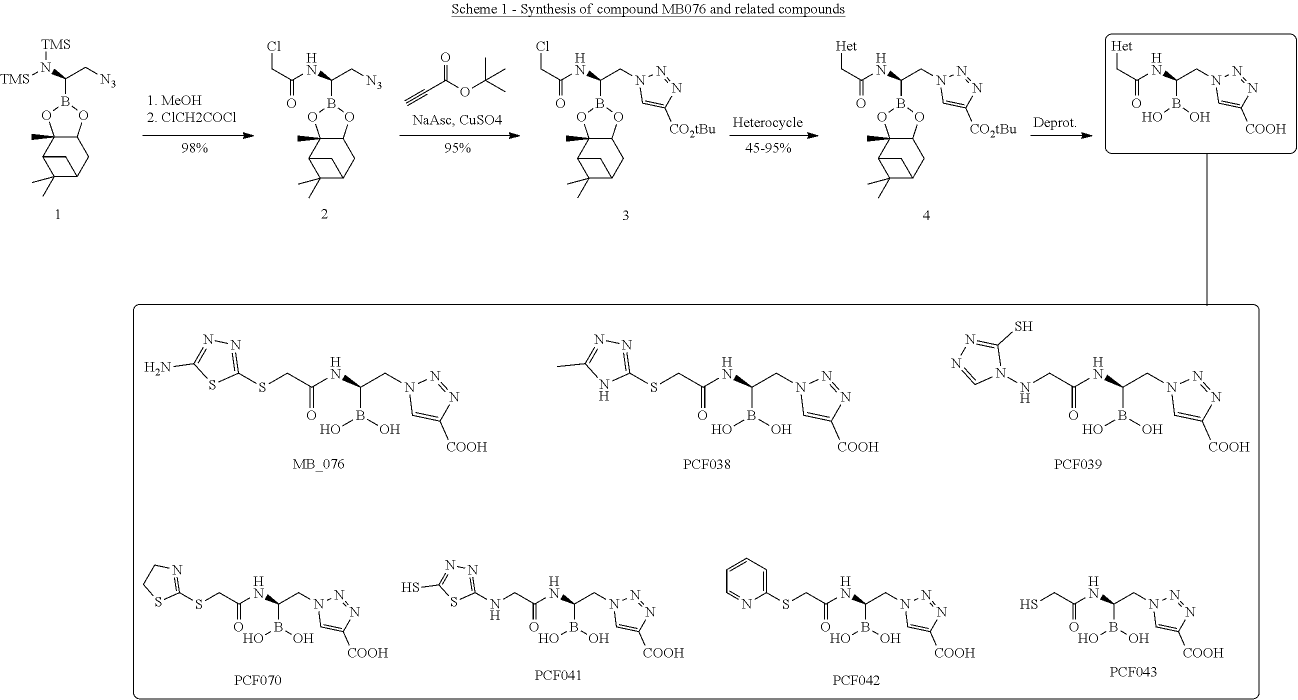

### Synthesis of MB054

The synthesis of MB054 started from compound 5 (scheme 2), obtained as described in J. Med. Chem. 2010, 7852-7863. This compound was subjected to acidic removal of the two TMS groups by treatment with a slight excess of HCl in dry dioxane at 0° C. After evaporation of the volatiles, the corresponding hydrochloride 6 was obtained in 37% yield as white crystals. Compound 6 was treated with chloroacetyl chloride and di-isopropyl-ethylamine in dichloromethane to afford compound 7, which was purified by column chromatography and isolated in 98% yield. Following the procedure described for MB076, nucleophilic displacement of the chlorine atom by 5-amino-1,3,4-thiadiazole-2-thiol was performed in dry acetonitrile and in the presence of triethylamine at room temperature, affording 8 (92% yield).

Final deprotections of the carboxylic and boronic groups were performed following the procedure already described for MB076, affording MB054 in 62% overall yield.

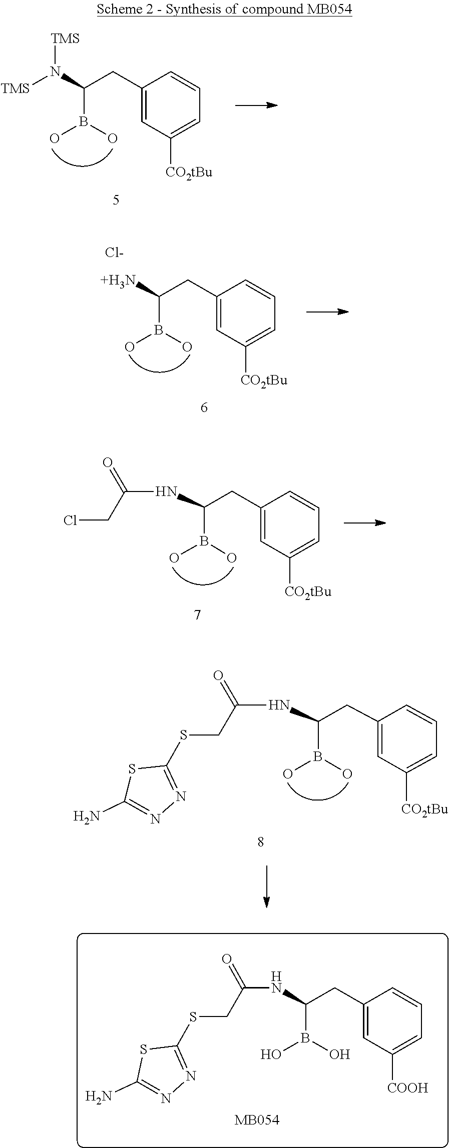 [SMILES: CC(C)(C)OC(=O)c1cccc(C[C@@H](B(O)O)N([Si](C)(C)C)[Si](C)(C)C)c1 ; [Cl-] ; CC(C)(C)OC(=O)c1cccc(C[C@H]([NH3+])B(O)O)c1 ; CC(C)(C)OC(=O)c1cccc(C[C@H](NC(=O)CCl)B(O)O)c1 ; CC(C)(C)OC(=O)c1cccc(C[C@H](NC(=O)CSc2nnc(N)s2)B(O)O)c1 ; Nc1nnc(SCC(=O)N[C@@H](Cc2cccc(C(=O)O)c2)B(O)O)s1]

### Synthesis of PCF013

PC15 was suspended in dry DCM and (Z)-2-(5-amino-1,2,4-thiadiazol-3-yl)-2-(((1-(tert-butoxy)-2-methyl-1-oxopropan-2-yl)oxy)imino)acetic acid (9, scheme 3) was added as a solid at 0° C. The reaction mixture was stirred 2.5 h and then DCM was removed in vacuo. The crude was then dissolved in dry DCM and the solvent was removed again affording the crude acid chloride 10, which was used as such for the next step.

To a freshly prepared (+)-pinanediol N,N-bis(trimethylsislyl)amino]-methaneboronate 11 (PNAS 2012, 109(43), 17448-17453) at 0° C. in THF, dry MeOH (1 eq.) was added one portion. The reaction mixture was stirred 0.5 h and 1.5 h at room temperature, the temperature was lowered to −40° C. and the acid chloride 10 in THF was added dropwise. The reaction mixture was warmed to 0° C. during 2.5 h. The solvent was then removed in vacuo, and the residue dissolved in ethyl acetate and washed with NaHCO₃. The organic phase was dried, and the solvent removed in vacuo to give a brownish wax. This residue was purified by column chromatography (silica gel, firstly pure Et₂O then pure EtOAc) and by crystallization from diethyl ether/cyclohexane to give 12 as a white solid (15% yield).

Final deprotections of the carboxylic and boronic functionalities were performed following the procedure already described for MB076, affording PCF013 in 61% overall yield.

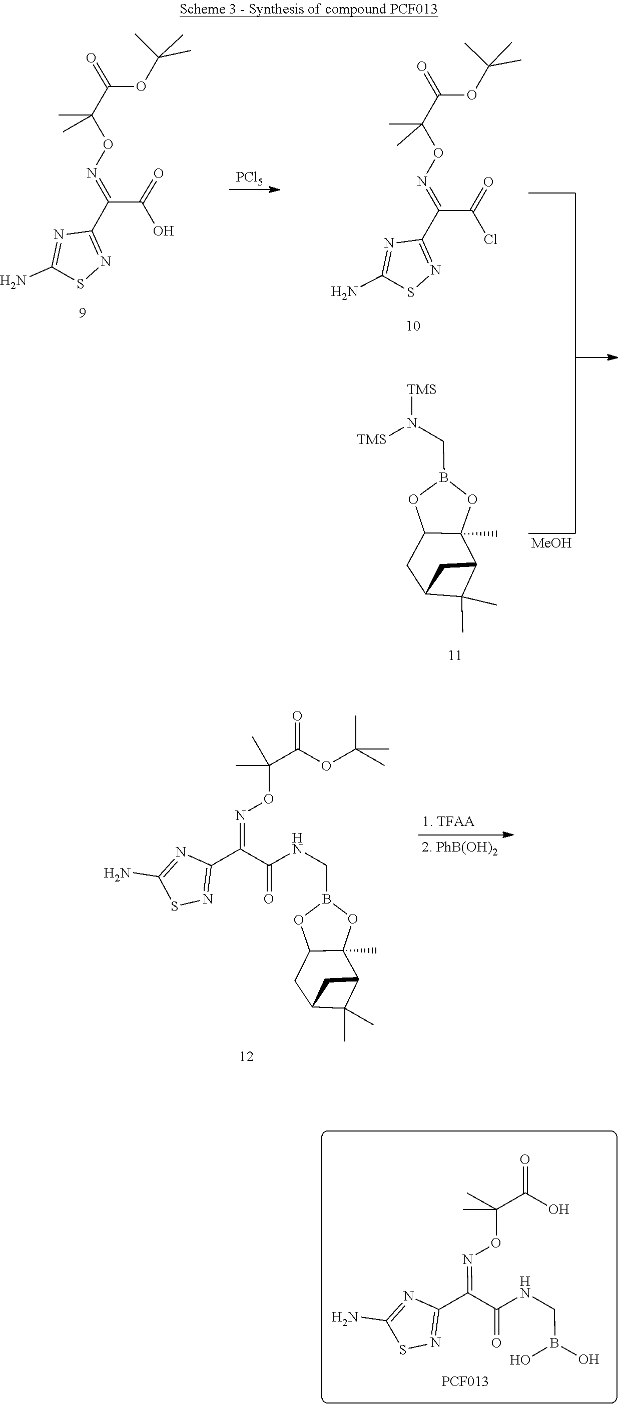

### Synthesis of PCF014

Anhydrous methanol (1 eq) was added at 0° C. to a solution of compound 1 (1 eq, scheme 4) in anhydrous THF and allowed to react at room temperature for 1 h. Thereafter the temperature was cooled again at 0° C. and a solution of 10 (1.1 eq) in anhydrous THF was slowly added; the cooling bath was removed, and the mixture stirred for additional 2 h. The solvent was removed, and the residue was dissolved in ethyl acetate, washed twice with brine and dried. The solvent was removed under reduced pressure and the residue purified by silica gel column chromatography (light petroleum/diethyl ether), affording compound 13 in 10% yield. Copper-catalyzed azide alkyne cycloaddition was performed with propiolic acid and 13 according to the procedure already described for the synthesis of MB076; after crystallization of the crude from diethyl ether/hexane the expected b-triazolylboronate 14 was obtained in 60% yield. Deprotection of the carboxylic and boronic functionalities were performed following the procedure already described for MB076, affording PCF014 in 69% overall yield.

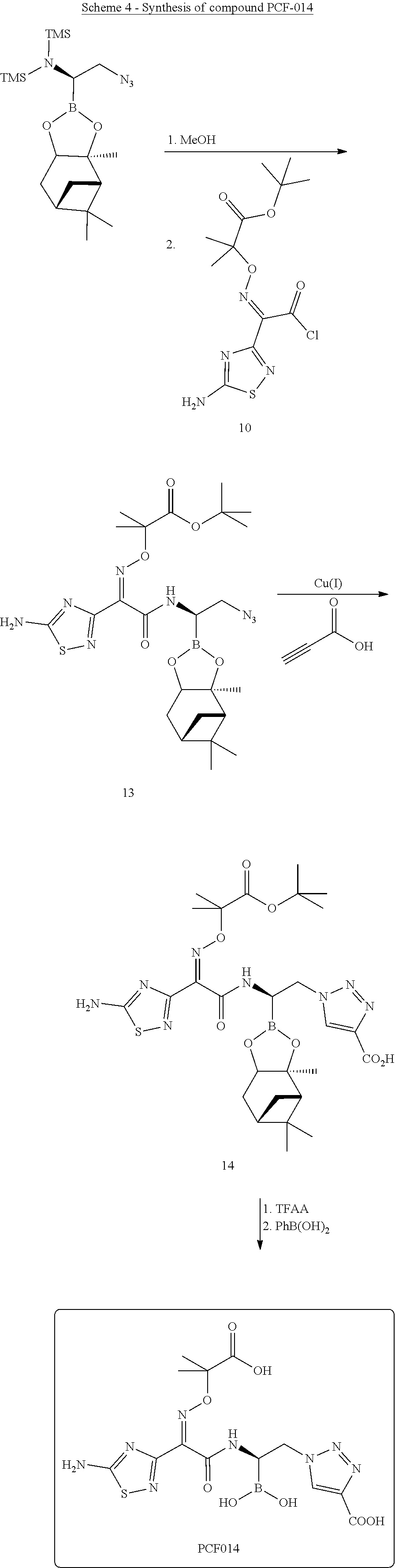

### Synthesis of PCF044

Compound 2 (scheme 5) was subjected to copper-catalyzed azide alkyne cycloaddition with (propioloyloxy)methyl 2-ethylbutanoate in the presence of CuSO₄ and sodium ascorbate (as described in J. Med. Chem. 2015, 545-5458) affording compound 15 in 86% yield.

To a solution compound 15 (1 eq) and triethylamine (1.05 eq) and in dry acetonitrile, 5-amino-1,3,4-thiadiazole-2-thiol (1.04 eq) was added and allowed to react for 3 hours. The mixture was concentrated in vacuo and partitioned between ethyl acetate and water and the aqueous phase extracted 3 times with ethyl acetate. The combined organic phases were dried, filtered, and concentrated to give the crude product which was triturated with diethyl ether and washed with hexane affording the desired product 16 in 63% yield. Final removal of (+)-pinanediol by transesterification with isobutylboronic acid afforded PCF044 in nearly quantitative amount.

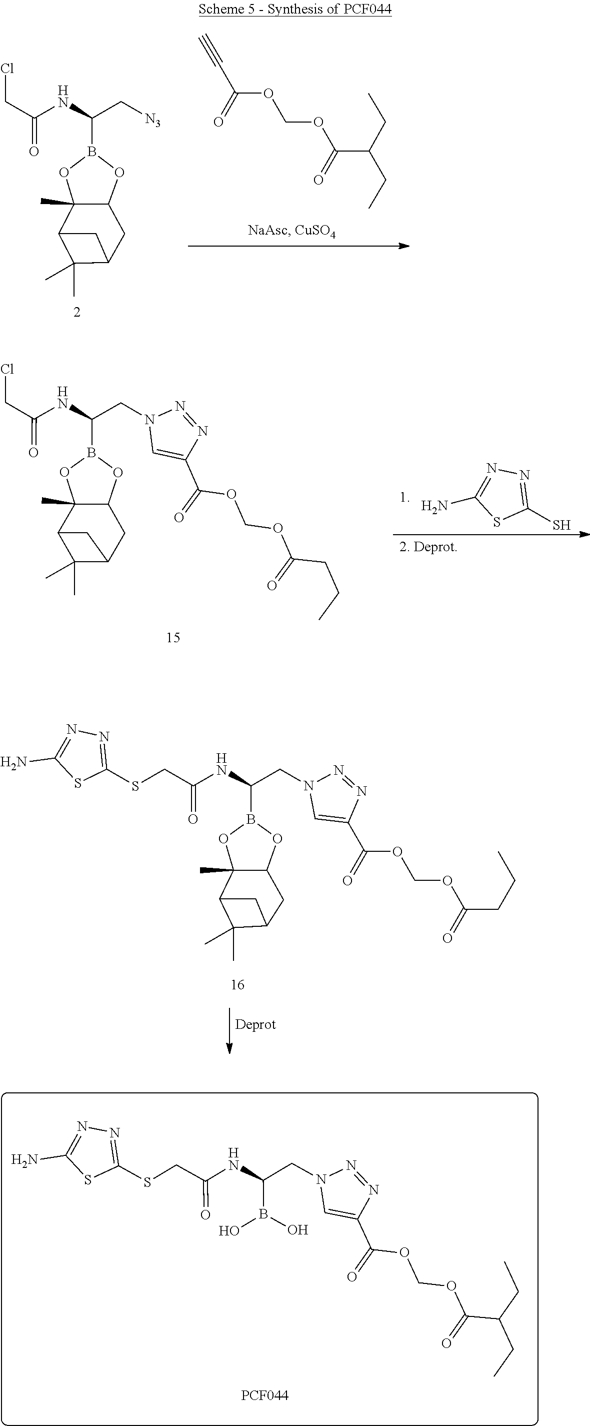

### EXPERIMENTAL

### Inhibition Kinetics for MB076 and Derivatives (PCF_038-043)

The binding affinities for compounds MB076, PCF_038-043 were calculated from competition assays using nitrocefin (NCF) as a reporter substrate. Nitrocefin was adjusted according to the Km (≈5×KM). The IC₅₀ values for all BATSIs, corrected for the NCF affinity are reported. Inhibitory activity was measured after 5 min incubation

TABLE 1

| | IC₅₀ vs | IC₅₀ vs | | IC₅₀ vs | IC₅₀ vs |
|---|---|---|---|---|---|
| | KPC-2 | PDC-3 | | KPC-2 | PDC-3 |
| Cmpd | (mM) | (mM) | Cmpd | (mM) | (mM) |
| MB076 | 0.135 | vs ADC-7= | MB054 | | |
| PCF_038 | 0.18 | 1.3 | PCF_041 | 0.46 | 0.63 |
| PCF_039 | 0.26 | 1.13 | PCF_042 | 0.58 | 0.79 |
| PCF_040 | 0.56 | 1.43 | PCF_043 | 0.17 | 1.13 |

TABLE 2
Inhibition Kinetics for “ceftolozane-like”
BATSIs (PCF_013-020 and S21 compounds)

| | IC₅₀ vs ADC-7 | IC₅₀ vs PDC-3 | Ki vs PDC-3 |
|---|---|---|---|
| Cmpd | (mM) | (mM) | (mM) |
| S21_205 | 3.5 | 0.6 | 0.12 |
| S21_230 | 0.5 | 0.2 | 0.04 |
| S21_225 | 9 | 2 | 0.4 |
| PCF_013 | | | |
| PCF_019 | | | |
| PCF_017 | | | |
| PCF_016 | | | |
| PCF_015 | | | |
| S21_226 | 0.3* 8 | 2.3 | 0.46 |
| S21_232 | 2.0* (25) | >20 | ND |
| PCF_020 | | | |
| PCF_014 | | | |

Minimum inhibitory concentration (MIC) of MB076 and derivatives (PCF_038-043) in combination with cefepime.

MICs were determined by the Agar microdilution method, following CLSI guidelines. Compounds were tested at fixed concentration of 4 ug/ml in combination with cefepime.

| Isolate | B-lactamases | Cefepime | PCF_038 | PCF_039 |
|---|---|---|---|---|
| E. coli DH10B pBC-SK control | none | <0.25 | <0.25 | <0.25 |
| E. coli ATCC BAA202 | SHV-1 | 1 | 0.5 | <0.25 |
| E. coli DH10B pBC-SK SHV-2 | SHV-2 | 4 | <0.25 | <0.25 |
| E. coli DH10B pBC-SK P99 | P99 | <0.25 | <0.25 | <0.25 |
| E. coli DH10B pBC-SK PDC-3 | PDC-3 | <0.25 | <0.25 | <0.25 |
| E. coli DH10B pBC-SK KPC-2 | KPC-2 | 8 | <0.25 | <0.25 |
| E. coli 29838 | CTX-M-14, TEM-1 | 4 | <0.25 | <0.25 |
| E. coli pLTCF1 | CTX-M group 9 | 8 | <0.25 | <0.25 |
| E. coli pLTCF2 | KPC?? | 1 | <0.25 | <0.25 |
| K. pneumoniae 34700 | CTX-M-15, SHV, TEM | 32 | 1 | 1 |
| K. pneumoniae [symbol] | TEM-1, SHV-5, CTX-M-2 | 1 | <0.25 | <0.25 |
| K. pneumoniae [symbol] | TEM-1, SHV-2, CTX-M-3 | 1 | <0.25 | <0.25 |
| K. pneumoniae 427 | TEM-1, SHV-1, CTX-M-3 | 8 | <0.25 | <0.25 |
| K. pneumoniae original KPC-2 | KPC-2 | 8 | <0.25 | 0.5 |
| K. pneumoniae ST17 | KPC-2, TEM, SHV, CTX-M-Group 1 | 64 | 32 | 16 |
| K. pneumoniae ST258 C3 | KPC-3 | 8 | 0.5 | <0.25 |
| K. pneumoniae ST258 B5 | KPC-2 | 32 | 16 | 4 |
| K. pneumoniae 253 | TEM-12, SHV-5, CTX-M | 32 | 2 | 1 |
| K. pneumoniae [symbol] | TEM-12, SHV-2, CTX-M-2 | 4 | <0.25 | <0.25 |
| K. pneumoniae [symbol] | TEM-1, SHV-5 | 1 | <0.25 | <0.25 |
| K. pneumoniae 266 | SHV-5 | 2 | <0.25 | <0.25 |
| K. pneumoniae [symbol] | TEM-1, SHV-1 | 0.5 | <0.25 | <0.25 |
| K. pneumoniae 255 | TEM-1, SHV-2 | 4 | 2 | 2 |
| A. baumannii 0057 | ADC-7 | 64 | 64 | 32 |

| Isolate | PCF_040 | PCF_041 | PCF_042 | PCF_043 | MB076 |
|---|---|---|---|---|---|
| E. coli DH10B pBC-SK control | <0.25 | <0.25 | <0.25 | <0.25 | <0.25 |
| E. coli ATCC BAA202 | 2 | 0.5 | <0.25 | 1 | <0.25 |
| E. coli DH10B pBC-SK SHV-2 | 0.5 | <0.25 | <0.25 | <0.25 | <0.25 |
| E. coli DH10B pBC-SK P99 | <0.25 | <0.25 | <0.25 | 1 | <0.25 |
| E. coli DH10B pBC-SK PDC-3 | <0.25 | <0.25 | <0.25 | <0.25 | <0.25 |
| E. coli DH10B pBC-SK KPC-2 | 1 | <0.25 | [symbol] | 1 | <0.25 |
| E. coli 29838 | 0.5 | <0.25 | <0.25 | 0.5 | <0.25 |
| E. coli pLTCF1 | 0.5 | 0.5 | <0.25 | 1 | <0.25 |
| E. coli pLTCF2 | <0.25 | <0.25 | <0.25 | <0.25 | <0.25 |
| K. pneumoniae 34700 | 4 | 2 | 1 | 4 | 1 |
| K. pneumoniae [symbol] | <0.25 | <0.25 | <0.25 | <0.25 | <0.25 |
| K. pneumoniae [symbol] | <0.25 | <0.25 | <0.25 | <0.25 | <0.25 |
| K. pneumoniae 427 | 1 | 0.5 | <0.25 | 1 | <0.25 |
| K. pneumoniae original KPC-2 | 2 | 0.5 | <0.25 | 1 | <0.25 |
| K. pneumoniae ST17 | 32 | 16 | 32 | 32 | 16 |
| K. pneumoniae ST258 C3 | 2 | 1 | 0.5 | 2 | 1 |
| K. pneumoniae ST258 B5 | 32 | 16 | 16 | 16 | 8 |
| K. pneumoniae 253 | 4 | 2 | [symbol] | 4 | 8 |
| K. pneumoniae [symbol] | 2 | 8 | <0.25 | 1 | 2 |
| K. pneumoniae [symbol] | <0.25 | 2 | <0.25 | <0.25 | <0.25 |
| K. pneumoniae 266 | <0.25 | <0.25 | <0.25 | <0.25 | <0.25 |
| K. pneumoniae [symbol] | <0.25 | <0.25 | <0.25 | <0.25 | <0.25 |
| K. pneumoniae 255 | 2 | | 2 | 4 | 2 |
| A. baumannii 0057 | 32 | 64 | 64 | 32 | 64 |

MB076 was further evaluated alone or in combination with cefepime or with meropenem against more K. pneumoniae strains. Tigecycline was included as control. [symbol]
indicates data missing or illegible when filed

TABLE 4

| | MIC, μg/mL | | | | | |
|---|---|---|---|---|---|---|
| Species/Strain | | | | Meropenem + | Cefepime + | |
| ID | MB076 | Meropenem | Cefepime | MB076 | MB076 | Tigecycline |
| Klebsiella | >32 | 32 | >32 | 0.06 / 4 | 0.25 / 4 | 2 |
| pneumonia/ | | | | | | |
| BAA-1705 | | | | | | |
| Klebsiella | >32 | >32 | >32 | 1 / 4 | 2 / 4 | 4 |
| pneumonia/ | | | | | | |
| FDA-CDC AR- | | | | | | |
| BANK#0097 | | | | | | |
| Klebsiella | >32 | 16 | 32 | 0.03 / 4 | ≤0.03 / 4 | 1 |
| pneumonia/ | | | | | | |
| FDA-CDC AR- | | | | | | |
| BANK#0098 | | | | | | |

Minimum Inhibitor Concentration (MIC) of “ceftolozane-like” BATSIs (PCF_013-020 and S21 compounds) in combination with different antibiotics (cefepime-FEP; ceftazidime-CAZ; sulbactam-SULB.

TABLE 5
MIC values [mg/L] of antibiotics in
combination with 10 μg/mL of compounds

| | K. pneum | K. pneum | | | M9 | |
|---|---|---|---|---|---|---|
| Compounds | bla_{KPC-2} | KPC-Kpn | bla_{CTXM-9} | bla_{ADC-7} | clinical | bla_{CMY-2} |
| Antibiotic | FEP | FEP 32 | FEP 8 | CAZ 16 | CAZ >32 | CAZ 128 |
| (MICmg/mL) | 16/32 | | | | | |
| S21_205 | 2 | 4 | | 4 | 16 | |
| S21_230 | 4/8 | 8 | | 2/4 | 4/8 | |
| S21_225 | 1 | 1 | | 16 | 4/8 | |
| PCF_013 | 16 | 16 | 4 | 4 | | 64 |
| PCF_019 | | | | | | |
| PCF_017 | 32 | 16 | 4 | 8 | | 64 |
| PCF_016 | 16 | 8 | 2 | 2 | | 64 |
| PCF_015 | 32 | 16 | 4 | 2 | | 64 |
| S21_226 | | | | | | |
| S21_232 | 8 | 16 | | 16 | 16 | |
| PCF_020 | 8 | 16 | | 16 | 16 | |
| PCF_014 | 32 | 32 | 2 | 8 | | 64 |

| MIC values [mg/L] of antibiotics in | | | | | |
|---|---|---|---|---|---|
| combination with 10 μg/mL of compounds | | | | | |
| | | | AB0057 | HUMC1 | NDM-1 |
| Compounds | 18SH | bla_{PDC-3E221K} | clinical | Abspp | pHSG298 |
| Antibiotic | CAZ 64 | CAZ 32 | SULB 32 | SULB 4 | IMI 8 |
| (MICmg/mL) | | | | | |
| S21_205 | | | 8 | 4 | 8 |
| S21_230 | | | 8/16 | 2 | 8 |
| S21_225 | | | 4 | 4/8 | 8 |
| PCF_013 | 64 | 8 | | | |
| PCF_019 | | | | | |
| PCF_017 | 64 | 32 | | | |
| PCF_016 | 64 | 32 | | | |
| PCF_015 | 64 | 32 | | | 8 |
| S21_226 | | | | | |
| S21_232 | | | | 4 | 8 |
| PCF_020 | | | | 4 | 8 |
| PCF_014 | 64 | 32 | | | |

### Maximum Tolerable Dose (MTD) for MB076

A maximum tolerable dose (MTD) assessment was conducted to evaluate the tolerability of MB076 with meropenem and cilastatin combination. Neutropenic uninfected female ICR mice were used. Meropenem combined with cilastatin was subcutaneously (SC) administered followed by intravenous (IV) administration of MB076 within 10 min. Doses were administered four times (QID) with 6 h intervals (q6h) for one day. Animals were monitored for acute symptoms after the first dose administration and cage side observations were recorded for the subsequent QID doses. Moreover, the body weight of each animal was recorded daily. The most significant results are summarized in Table 6. No acute toxicity or mortality was observed in the tested dose groups.

TABLE 6

| | Body weight | Body weight | | Abdominal | Limb | |
|---|---|---|---|---|---|---|
| Dosage | after 1° dose | after 4° dose | Vocalization | tone | tone | Piloerection |
| MEM 1000 mg/Kg + | 23 | 23 | 2/4 | increased | Mild | 4/4 after |
| cilastatin 200 mg/Kg | | | | | effect | 4° dose |
| (SC) + vehicle (PBS, IV) | | | | | | |
| MEM 1000 mg/Kg + | 23 | 22 | 0/4 | Mild | Mild | 4/4 after |
| cilastatin 200 mg/Kg | | | | effect | effect/ | 4° dose |
| (SC) + MB076 100 | | | | | increased | |
| mg/Kg (IV) | | | | | | |
| MEM 1000 mg/Kg + | 23 | 22 | 3/4 | Mild | Mild | 4/4 after |
| cilastatin 200 mg/Kg | | | | effect | effect | 4° dose |
| (SC) + MB076 50 | | | | | | |
| mg/Kg (IV) | | | | | | |
| MEM 1000 mg/Kg + | 23.2 | 21.7 | 2/4 | Mild | Mild | 4/4 after |
| cilastatin 200 mg/Kg | | | | effect | effect | 4° dose |
| (SC) + MB076 25 | | | | | | |
| mg/Kg (IV) | | | | | | |
| MEM 500 mg/Kg + | 23 | 22.2 | 0/4 | increased | Mild | 4/4 after |
| cilastatin 200 mg/Kg | | | | | effect | 4° dose |
| (SC) + MB076 50 | | | | | | |
| mg/Kg (IV) | | | | | | |
| MEM 500 mg/Kg + | 23 | 22 | 1/4 | Mild | Mild | 4/4 after |
| cilastatin 200 mg/Kg | | | | effect | effect | 4° dose |
| (SC) + MB076 25 | | | | | | |
| mg/Kg (IV) | | | | | | |

### In Vivo Efficacy Study of MB076

To determine in vitro activity of MB076 we used cefepime with or without MB076 in mice infected IV with the pan drug resistant strain KP1 and treated IP at the time of infection with placebo control, cefepime, or cefepime+MB076 (at 1:4 ratio). Doses were repeated the following morning and afternoon (18 h and 24 h after infection, respectively). All mice treated with placebo or cefepime alone died, whereas all mice treated with either ratio of MB076 plus cefepime survived. Results showed that mice treated with placebo or cefepime alone died, whereas all mice treated with MB076 plus cefepime 1:4 survived

### Pharmacokinetic and Pharmacodynamic Profile of MB076

A single dose PK study with MB_076 has been conducted to evaluate antibiotic exposure over time in plasma samples and thigh homogenates following a single dose IV administration in K. pneumoniae AR-BANK #0098 (KPC-2) thigh infection model with neutropenic female ICR mice. Among the PK parameters, t_{1/2}, C₀, AUCₗₐₛₜ, AUC_{INF}, Vss and CL were evaluated (Table 7), while for PD parameters, C₀/MIC, AUCₗₐₛₜ/MIC and T>MIC were calculated (Table 8). In particular, the % time over the minimum inhibitory concentration (T>MIC) was calculated for all dose groups to determine the impact of MB076 efficacy in this animal model. Plots of concentration in plasma and thigh homogenate versus time were created and the linearity of the resulting PK parameters was assessed. Indeed, the concentration time profile of MB076 yielded C₀ and AUCₗₐₛₜ values that correlated linearly with dose both in plasma and in tight homogenates.

TABLE 7
The plasma and thigh PK parameters of MB076 after single IV administration
to K. pneumoniae AR-BANK#0098 (KPC-2) thigh infected mice

| Dose, IV | Time point | t_{1/2} | C₀ | AUCₗₐₛₜ | AUC_{Inf} | Vss | CL |
|---|---|---|---|---|---|---|---|
| (mg/kg) | (h) | (h) | (μg/mL) | (h*μg/mL) | (h*μg/mL) | (L/kg) | (mL/min/kg) |
| 12.5 | 0.083-4.0 | 0.56 | 35.6 | 7.1 | 7.2 | 0.65 | 29.05 |
| 25 | 0.083-4.0 | 0.61 | 84.0 | 17.6 | 17.8 | 0.80 | 23.42 |
| 50 | 0.083-4.0 | 0.43 | 206.9 | 39.0 | 39.1 | 0.35 | 21.31 |
| 100 | 0.083-4.0 | 0.43 | 638.1 | 103.2 | 103.5 | 0.21 | 16.11 |
| Dose, IV | Time point | t_{1/2} | C₀ | AUCₗₐₛₜ | AUC_{Inf} | Vss | CL |
| (mg/kg) | (h) | (h) | (μg/mL) | (h*μg/mL) | (h*μg/mL) | (L/kg) | (g/min/kg) |
| 12.5 | 0.083-4.0 | 0.24 | 6.3 | 1.0 | 1.0 | 2.96 | 201.07 |
| 25 | 0.083-4.0 | 0.11 | 17.0 | 1.7 | 1.8 | 1.38 | 231.77 |
| 50 | 0.083-4.0 | 0.21 | 34.0 | 6.3 | 6.3 | 1.76 | 131.74 |
| 100 | 0.083-4.0 | 0.25 | 107.6 | 17.2 | 17.3 | 1.07 | 96.46 |

TABLE 8
The plasma and thigh PD parameters of MB076 after
single IV administration to K. pneumoniae AR-
BANK#0098 (KPC-2) thigh infected mice

| | Time | Time | Dosage | T > | | AUCₗₐₛₜ/ |
|---|---|---|---|---|---|---|
| Dose, IV | Low | High | interval | MIC | C₀/ | MIC |
| (mg/kg) | (h) | (h) | (h) | (%) | MIC | (h) |
| 12.5 | 23.60 | 0.40 | 24 | 1.69 | 8.91 | 1.77 |
| 25 | 23.42 | 0.58 | 24 | 2.43 | 21.01 | 4.41 |
| 50 | 23.07 | 0.93 | 24 | 3.89 | 51.73 | 9.75 |
| 100 | 22.92 | 1.08 | 24 | 4.51 | 159.52 | 25.81 |
| 12.5 | 23.94 | 0.06 | 24 | 0.24 | 1.57 | 0.25 |
| 25 | 23.88 | 0.12 | 24 | 0.50 | 4.26 | 0.43 |
| 50 | 23.58 | 0.42 | 24 | 1.75 | 8.51 | 1.56 |
| 100 | 23.35 | 0.65 | 24 | 2.72 | 26.89 | 4.30 |

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

1. A compound having the structure of formula (I): 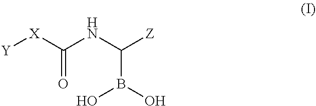
or a pharmaceutically acceptable salt, tautomer, or solvate thereof, wherein:
X is alkylene or CNOR¹;
Y is —W—R² or R²;
Z is H, -alkylene-aryl, -alkylene-heterocyclyl, or -alkylene-heteroaryl, each of which is optionally substituted with one or more R³;
W is S, N, O, S(O)ₘN, or alkylene optionally substituted with one or more halogen, alkyl, —COOH, —NH₂, or —OH;
R¹ is alkyl, -alkylene-OH optionally substituted with —OH, -alkylene-COOH optionally substituted with —OH, -alkylene-O-alkylene-OH, —C(O)-alkyl, —C(O)O-alkyl, or —C(O)O-alkylene-O—C(O)-alkyl;
R² is heterocyclyl or heteroaryl, each of which is optionally substituted with one or more R⁴;
each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), or —S(O₂)O⁻;
each R⁴ is independently halogen, alkyl, —COOH, —NH₂, or —OH;
each R⁵ is independently H or alkyl;
m is 0, 1, or 2; and
if X is CH₂, Y is —W—R².

2. The compound of claim 1, wherein R¹ is C₁-C₆ alkyl, —(C₁-C₆ alkylene)-OH optionally substituted with —OH, —(C₁-C₆ alkylene)-COOH optionally substituted with —OH, —(C₁-C₆ alkylene)-O—(C₁-C₆ alkylene)-OH, —C(O)—(C₁-C₆ alkyl), —C(O)O—(C₁-C₆ alkyl), or —C(O)O—(C₁-C₆ alkylene)-O—C(O)—(C₁-C₆ alkyl).

3. The compound of claim 1, wherein R¹ is C₁-C₆ alkyl or —(C₁-C₆ alkylene)-COOH optionally substituted with —OH.

4. The compound of claim 1, wherein W is an S, N, O, or S(O)ₘN; and m is 0, 1, or 2.

5. The compound of claim 1, wherein R² is 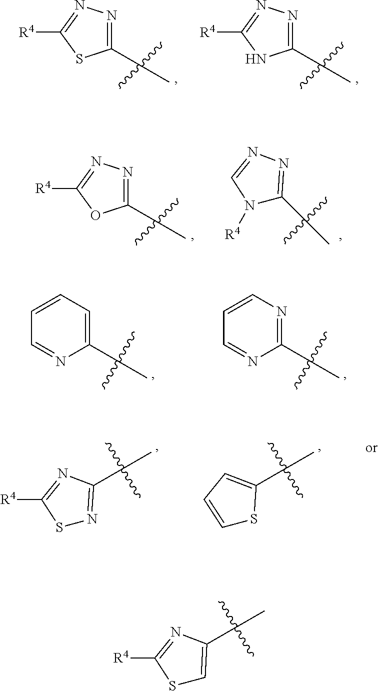
and R⁴ is halogen, alkyl, —COOH, —NH₂, —OH, or absent.

6. The compound of claim 1, wherein Z is H, —CH₂-5- to 6-membered aryl, —CH₂-5- to 6-membered heterocyclyl, —CH₂-5- to 6-membered heteroaryl, each of which is optionally substituted with one or more R³.

7. The compound of claim 1, wherein each R³ is independently 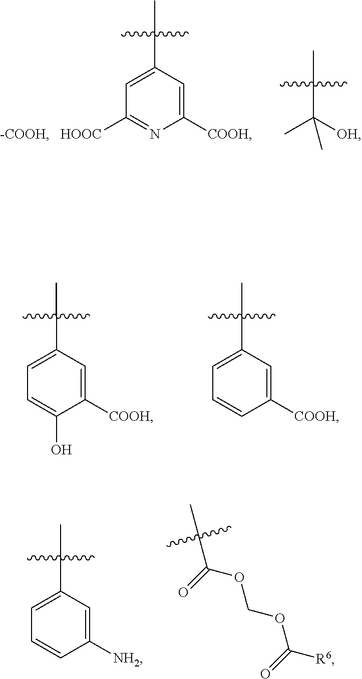
wherein R⁶ is alkyl, optionally substituted with one or more halogen, —COOH, —NH₂, or —OH.

8. The compound of claim 1, wherein, Z is H, 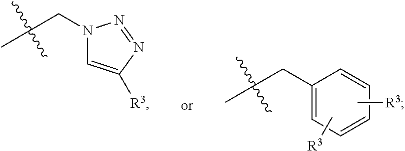
and
each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), or —S(O₂)O⁻ or absent; and each R⁵ is independently H or alkyl.

9. A compound having the structure of formula (II): 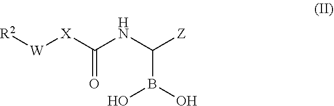
or a pharmaceutically acceptable salt, tautomer, or solvate thereof, wherein:
X is alkylene or CNOR¹;
Z is H, -alkylene-aryl, -alkylene-heterocyclyl, or -alkylene-heteroaryl, each of which is optionally substituted with one or more R³;
W is S, N, O, S(O)ₘN, or alkylene optionally substituted with one or more halogen, alkyl, —COOH, —NH₂, or —OH;
R¹ is alkyl, -alkylene-OH optionally substituted with —OH, -alkylene-COOH optionally substituted with —OH, -alkylene-O-alkylene-OH, —C(O)-alkyl, —C(O)O-alkyl, or —C(O)O-alkylene-O—C(O)-alkyl;
R² is heterocyclyl or heteroaryl, each of which is optionally substituted with one or more R⁴;
each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), or —S(O₂)O⁻;
each R⁴ is independently halogen, alkyl, —COOH, —NH₂, or —OH;
each R⁵ is independently H or alkyl; and
m is 0, 1, or 2.

10. The compound of claim 9, wherein W is an S, N, O, or S(O)ₘN; and m is 0, 1, or 2.

11. The compound of claim 9, wherein R¹ is C₁-C₆ alkyl, —(C₁-C₆ alkylene)-OH optionally substituted with —OH, —(C₁-C₆ alkylene)-COOH optionally substituted with —OH, —(C₁-C₆ alkylene)-O—(C₁-C₆ alkylene)-OH, —C(O)—(C₁-C₆ alkyl), —C(O)O—(C₁-C₆ alkyl), or —C(O)O—(C₁-C₆ alkylene)-O—C(O)—(C₁-C₆ alkyl).

12. The compound of claim 9, wherein R¹ is C₁-C₆ alkyl or —(C₁-C₆ alkylene)-COOH optionally substituted with —OH.

13. The compound of claim 9, wherein R² is: 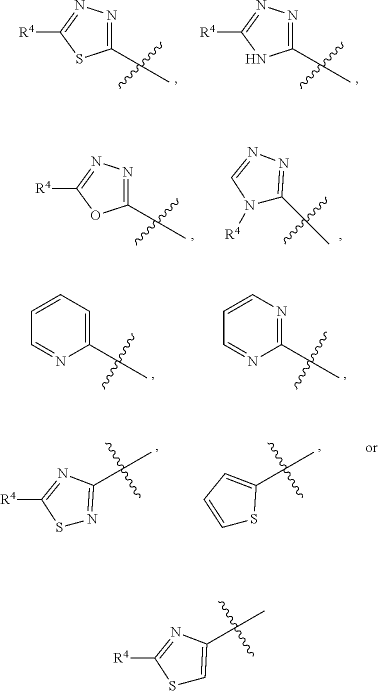
and R⁴ is halogen, alkyl, —COOH, —NH₂, —OH, or absent.

14. The compound of claim 9, wherein Z is H, —CH₂-5- to 6-membered aryl, —CH₂-5- to 6-membered heterocyclyl, or —CH₂-5- to 6-membered heteroaryl, each of which is optionally substituted with one or more R³.

15. The compound of claim 9, wherein each R³ is independently 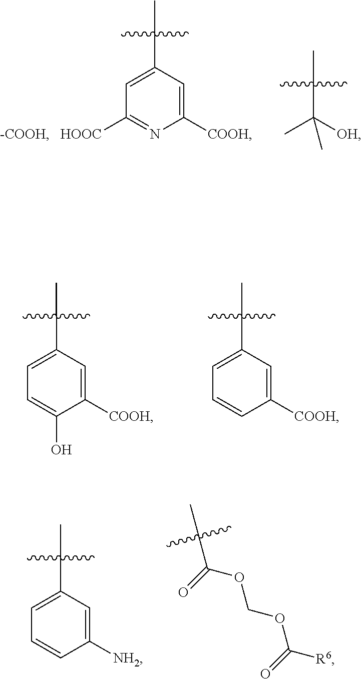
wherein R⁶ is alkyl, optionally substituted with one or more halogen, —COOH, —NH₂, or —OH.

16. The compound of claim 9, wherein, Z is H, 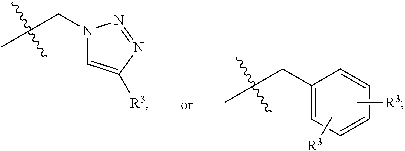
and
each R³ is independently alkyl, halogen, —N(R⁵)₂, —OH, —COOH, -alkylene-OH optionally substituted with —OH, -alkylene-COOH optionally substituted with —OH, -alkylene-O-alkylene-OH, —C(O)-alkyl, —C(O)O-alkyl, —C(O)O-alkylene-O—C(O)-alkyl, alkylene-(C(O)O⁻)C(O)O⁻, aryl optionally substituted with one or more halogen, alkyl, —COOH, —NH₂, or —OH, heterocyclyl optionally substituted with one or more halogen, alkyl, —COOH, —NH₂, or —OH, heteroaryl optionally substituted with one or more halogen, alkyl, —COOH, —NH₂, or —OH, —P(O)(O⁻)₂, —P(O)(O⁻), —S(O₂)O⁻ or absent; and each R⁵ is independently H or alkyl

17-30. (canceled)

31. The compound of claim 1 having a structure of: 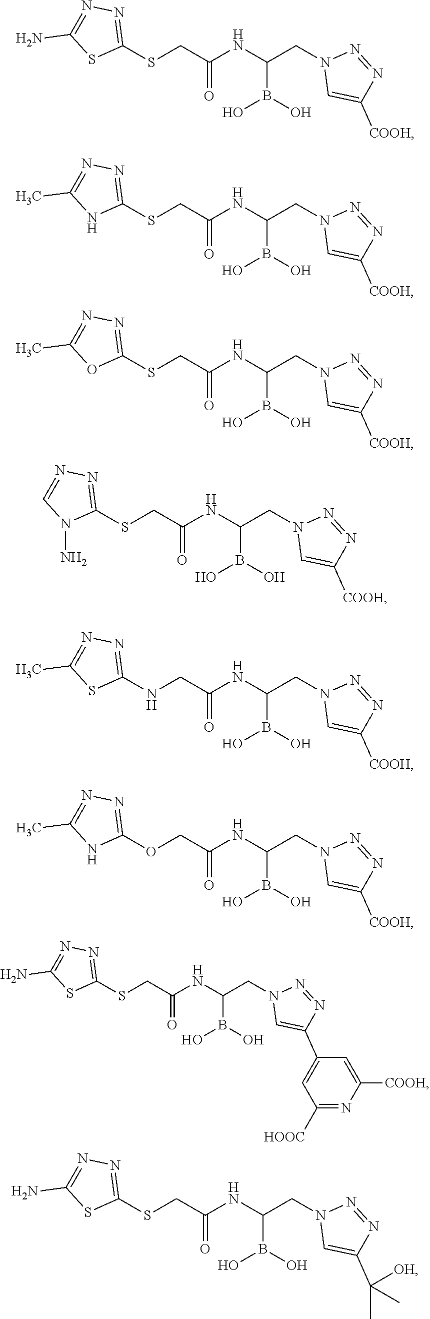 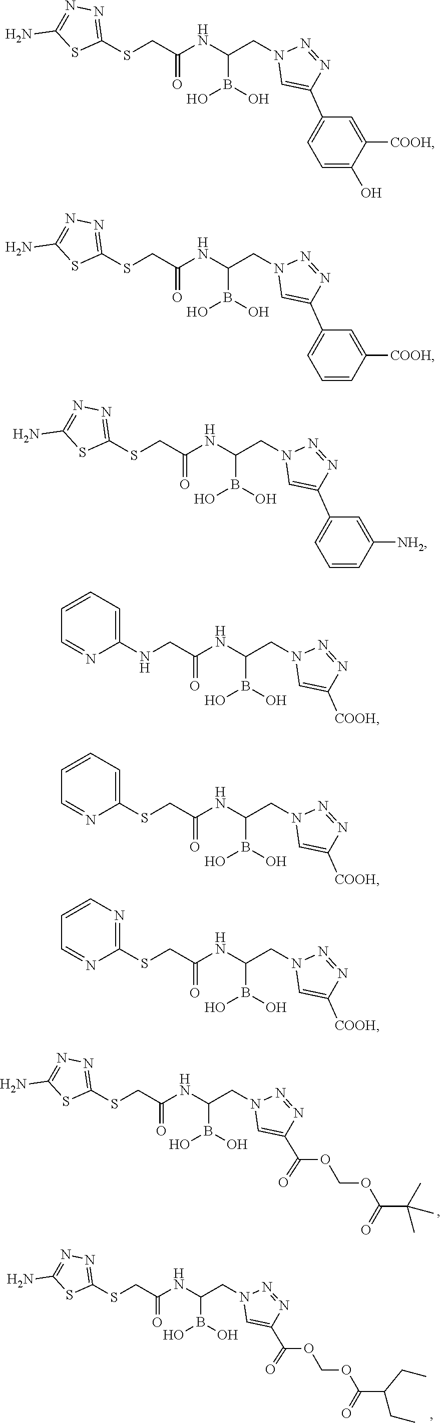 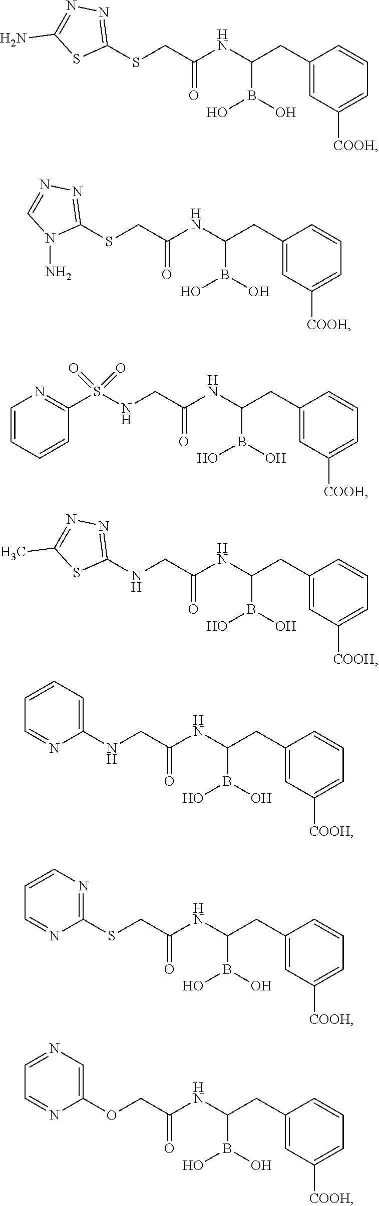 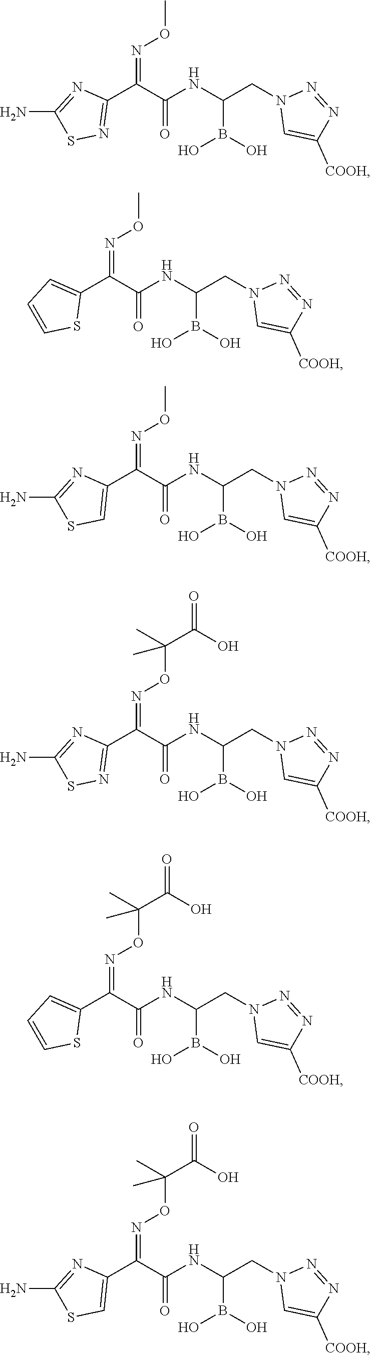 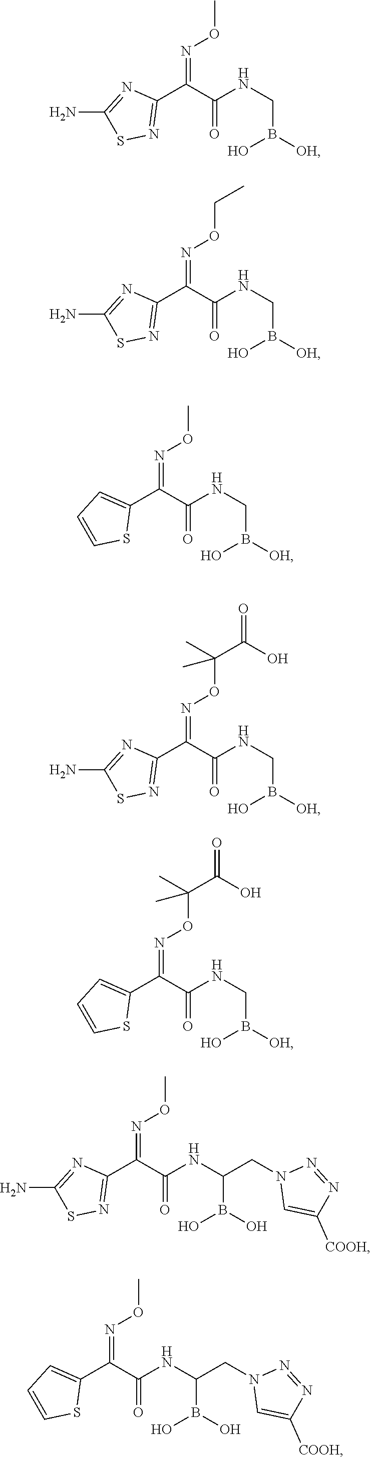 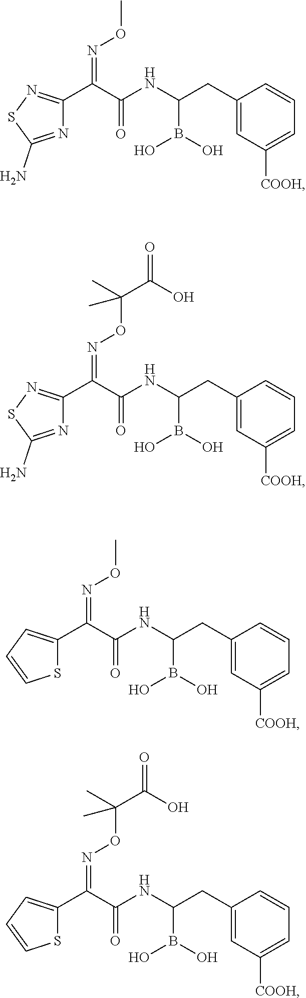 [SMILES: CO/N=C(\C(=O)NC(Cc1cccc(C(=O)O)c1)B(O)O)c1nsc(N)n1 ; CC(C)(O/N=C(\C(=O)NC(Cc1cccc(C(=O)O)c1)B(O)O)c1nsc(N)n1)C(=O)O ; CO/N=C(\C(=O)NC(Cc1cccc(C(=O)O)c1)B(O)O)c1cccs1 ; CC(C)(O/N=C(\C(=O)NC(Cc1cccc(C(=O)O)c1)B(O)O)c1cccs1)C(=O)O]
or a pharmaceutically acceptable salt, tautomer, or solvate thereof.

32. The compound of claim 1, not having the structure:

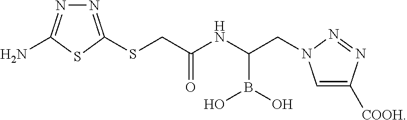 [SMILES: Nc1nnc(SCC(=O)NC(Cn2cc(C(=O)O)nn2)B(O)O)s1]

33. A pharmaceutical composition comprising a compound of claim 1 and a β-lactam antibiotic.

34. The pharmaceutical composition of claim 33, wherein the β-lactam antibiotic comprises at least one of a penicillin, cephalosporin, penem, carbapenem, or monobactam.

35-47. (canceled)
